(19) United States
(12) Patent Application Publication
(10) Pub. No.: US 2021/0114006 A1
(43) Pub. Date: Apr. 22, 2021

(54) BI-FUNCTIONAL CATALYST FOR THE PRODUCTION OF OLEFINS FROM SYNTHESIS GAS

(71) Applicant: BASF SE, Ludwigshafen am Rhein (DE)

(72) Inventors: Christiane KURETSCHKA, Ludwigshafen am Rhein (DE); Robert MCGUIRE, Florham Park, NJ (US); Ivana JEVTOVIKJ, Heidelberg (DE); Sven REINING, Ludwigshafen am Rhein (DE); Stefan LIPP, Ludwigshafen am Rhein (DE); Andrei-Nicolae PARVULESCU, Ludwigshafen am Rhein (DE); Astrid Elisa NIEDERLE, Ludwigshafen am Rhein (DE); Michael GESKE, Berlin (DE)

(21) Appl. No.: 17/254,484

(22) PCT Filed: Jun. 19, 2019
(86) PCT No.: PCT/EP2019/066202

§ 371 Date: Dec. 21, 2020

(30) Foreign Application Priority Data
Jun. 20, 2018 (EP) 18178703.7

(51) Int. Cl. B01J 29/70; B01J 23/26; B01J 37/02; B01J 37/00; B01J 37/03; B01J 37/06; B01J 37/08; C07C 1/04; C07C 29/153; C07C 41/01

(52) U.S. Cl. CPC B01J 29/70; B01J 23/26; B01J 37/0201; B01J 37/0018; B01J 37/031; B01J 37/06; C07C 2523/26; B01J 37/088; C07C 1/043; C07C 29/153; C07C 41/01; C07C 2529/70; B01J 37/0236

(57) ABSTRACT

The present invention relates to a composition comprising a) a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM; and b) a mixed metal oxide comprising chromium, zinc, and aluminum; and to a process for its production, as well as to the molding and the mixed metal oxide as such, respectively, as obtainable or obtained according to the inventive production process, as well as to the composition as obtainable or obtained according to the inventive production process. In addition to these, the present invention further relates to the use of the inventive composition as a catalyst or as a catalyst component, as well as to a process for preparing C2 to C4 olefins from a synthesis gas comprising hydrogen and carbon monoxide.

### TECHNICAL FIELD

The present invention relates to a composition comprising a molding comprising a zeolitic material having an AEI-type framework structure and a mixed metal oxide comprising chromium, zinc, and aluminum, as well as to a process for its production. Furthermore, the present invention relates to the molding and the mixed metal oxide as such, respectively, as obtainable or obtained according to the inventive production process, as well as to the composition as obtainable or obtained according to the inventive production process. In addition to these, the present invention further relates to the use of the inventive composition as a catalyst or as a catalyst component, as well as to a process for preparing C2 to C4 olefins from a synthesis gas comprising hydrogen and carbon monoxide.

### INTRODUCTION

In view of increasing scarcity of mineral oil deposits which serve as a starting material for the preparation of lower hydrocarbons and derivatives thereof, alternative processes for preparing such commodity chemicals are becoming increasingly important. In alternative processes for obtaining lower hydrocarbons and derivatives thereof, specific catalysts are frequently used to obtain lower hydrocarbons and derivatives thereof, such as unsaturated lower hydrocarbons in particular, with maximum selectivity from other raw materials and/or chemicals. In this context, important processes include those in which methanol as a starting chemical is subjected to a catalytic conversion which can generally lead to a mixture of hydrocarbons and derivatives thereof, and also aromatics.

In the case of such catalytic conversions, the particular challenge is to refine the catalysts used therein, and also the process regime and parameters thereof, in such a way that a few very specific products are formed with maximum selectivity in the catalytic conversion. In the past few decades, particular significance has been gained by those processes which enable the conversion of methanol to olefins and are accordingly characterized as methanol-to-olefin processes (MTO). For this purpose, there has been development particularly of catalysts and processes which convert the conversion of methanol via the dimethyl ether intermediate to mixtures the main constituents of which are ethene and propene.

U.S. Pat. No. 4,049,573, for example, relates to a catalytic process for the conversion of lower alcohols and ethers thereof, and especially methanol and dimethyl ether, to obtain a hydrocarbon mixture with a high proportion of C2-C3-olefins and monocyclic aromatics and especially para-xylene.

Goryainova et al., Petroleum Chemistry, vol. 51, no. 3 (2011), pp. 169-173 describes the catalytic conversion of dimethyl ether to lower olefins using magnesium-containing zeolites.

Typically, syngas conversion to olefins occurs in separates steps. First the syngas is converted to methanol and in a second stage methanol is converted to olefins. Syngas conversion to methanol is equilibrium limited with typical one-pass COₓ conversion of 63%. Methanol is separated from unprocessed syngas and then converted to olefins. The so called Lurgi's methanol-to-propylene (MTP) process uses separate fixed-bed reactors to produce the intermediate compound dimethyl ether (DME) and olefins, whereas other processes rely on a fluidized-bed reactor for the methanol-to-olefin conversion. The reactor effluent of these processes contains a mixture of hydrocarbons (olefins, alkanes), which requires several purification steps. Wan, V. Y., Methanol to Olefins/Propylene Technologies in China, Process Economics Programm, 261A (2013) discloses that often, depending on the intended product spectrum, undesired compounds are recycled back to the olefin reactor (Lurgi process) or cracked in a separate stage to enhance yield (Total/UOP process).

In Li, J. et al., Chinese Journal of Catalysis, vol. 36, no. 7 (2015), pp. 1131-1135, further alternative technology to produce olefins from synthesis gas (syngas) has been proposed which combines the synthesis steps in a single reactor wherein the syngas is first converted to methanol which is then dehydrated to olefins via the intermediate dimethyl ether (DME).

Propylene consumption is growing and predicted to grow in the next years by more than 4% annually. There is hence the need of a process that produces propylene in a high amount, a high selectivity, and that is economically efficient.

Unpublished patent application EP 17185280.9 relates to a composition comprising a molding comprising a zeolitic material having a CHA-type framework structure and a mixed metal oxide comprising chromium, zinc, and aluminum, as well as to a process for its production. Furthermore, the aforementioned unpublished patent application relates to the molding and the mixed metal oxide as such, respectively, as obtainable or obtained according to the production process described therein, as well as to the composition as obtainable or obtained according to said production process. In addition to these, the aforementioned unpublished patent application further relates to the use of said composition as a catalyst or as a catalyst component, as well as to a process for preparing C2 to C4 olefins from a synthesis gas comprising hydrogen and carbon monoxide.

### DETAILED DESCRIPTION

In spite of the advances which have been achieved with respect to the selection of raw materials and the conversion products thereof which can be used for the production of olefins, there is still a need for novel processes and catalysts which give a higher efficiency for the conversion and selectivity. More particularly, there is a constant need for novel processes and catalysts which, proceeding from the raw materials, lead via a minimum number of intermediates very selectively to the desired end product. Furthermore, it is desirable for efficiency purposes to be enhanced further by development of processes which require a minimum number of workup steps for the intermediates in order that they can be used in the subsequent stage

Surprisingly, it was found that C2 to C4 olefins and particularly propylene is produced in high amount, high selectivity and in an economically efficient one step process by using a catalyst composition comprising a molding comprising a specific zeolitic material and a mixed metal oxide comprising chromium, zinc, and aluminium.

Therefore, the present invention relates to a composition comprising

a) a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM; and
b) a mixed metal oxide comprising chromium, zinc, and aluminum.

As regards the trivalent element X comprised in the framework structure of the zeolitic material, no particular restriction applies. It is preferred that the trivalent element X is selected from the group consisting of Al, B, In, Ga, and mixtures of two or more thereof, more preferably from the group consisting of Al, B and mixtures thereof, X more preferably being Al and/or B, and more preferably being Al.

As regards the one or more alkali metals AM comprised in the zeolitic material, no particular restriction applies. It is preferred that the one or more alkali metals AM is one or more of Li, Na, K, Rb, and Cs, wherein the one or more alkali metals AM more preferably comprises Na, more preferably the one or more alkali metals AM is Na.

Therefore, it is particularly preferred that the trivalent element X comprised in the framework structure of the zeolitic material is selected from the group consisting of Al, B, In, Ga, and mixtures of two or more thereof, more preferably from the group consisting of Al, B and mixtures thereof, X more preferably being Al and/or B, and more preferably being Al, wherein it is preferred that the one or more alkali metals AM is one or more of Li, Na, K, Rb, and Cs, wherein the one or more alkali metals AM more preferably comprises Na, more preferably the one or more alkali metals AM is Na.

As disclosed above, the framework structure of the zeolitic material comprises Si, a trivalent element X, and oxygen. As regards the molar ratio Si:X calculated as SiO₂:X₂O₃ of the framework structure of the zeolitic material, no particular restriction applies. It is preferred that the molar ratio Si:X calculated as SiO₂:X₂O₃ of the framework structure of the zeolitic material is in the range of from 4 to 300, and preferably in the range of from 6 to 150, more preferably from 8 to 100, more preferably from 10 to 50, more preferably from 11 to 30, more preferably from 12 to 20, and more preferably from 12.5 to 16. According to the present invention it is particularly preferred that the molar ratio Si:X calculated as SiO₂:X₂O₃ of the framework structure of the zeolitic material is in the range of from 13 to 14.

As regards the physical and/or chemical nature of the framework structure of the zeolitic material, no particular restriction applies such that further compounds and/or elements may be contained therein. It is preferred that at least 95 weight-%, preferably at least 98 weight-%, more preferably at least 99 weight-%, more preferably at least 99.5 weight-%, more preferably at least 99.9 weight % of the framework structure of the zeolitic material consist of Si, X, 0, and H.

As disclosed above, no particular restriction applies in view of the physical and/or chemical nature of the framework structure of the zeolitic material, such that further components, e. g. phosphorous, may be contained therein. It is preferred that at most 1 weight-%, preferably at most 0.1 weight-%, more preferably at most 0.01 weight-%, more preferably from 0 to 0.001 weight-% of the framework structure of the zeolitic material consist of phosphorous.

As disclosed above, the zeolitic material has a framework structure comprising Si, a trivalent element X, and oxygen, wherein the zeolitic material further comprises one or more alkali metals AM and/or one or more alkaline earth metals AEM. As regards the physical and/or chemical nature of the zeolitic material having a framework structure comprising Si, a trivalent element X, and oxygen, wherein the zeolitic material comprises one or more alkali metals AM and/or one or more alkaline earth metals AEM, again no particular restriction applies. It is preferred that at least 95 weight-%, preferably at least 98 weight-%, more preferably at least 99 weight-%, more preferably at least 99.5 weight-%, more preferably at least 99.9 weight-% of the zeolitic material consist of Si, X, O, H, and the one or more alkali metals AM and/or the one or more alkaline earth metals AEM.

As disclosed above, the molding comprises a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM. As regards the physical and/or chemical nature of the molding, no particular restriction applies such that further components, e. g. a binder material, may be comprised therein. It is preferred that the molding further comprises a binder material.

In the case where the molding further comprises a binder material, no particular restriction applies in view of the physical and/or chemical nature of the binder material. It is preferred that the binder material comprises, preferably is one or more of graphite, silica, titania, zirconia, alumina, and a mixed oxide of two or more of silicon, titanium, zirconium, and aluminum, wherein more preferably, the binder material comprises silica, more preferably is silica.

Therefore, it is particularly preferred that the molding further comprises a binder material, wherein the binder material preferably comprises one or more of graphite, silica, titania, zirconia, alumina, and a mixed oxide of two or more of silicon, titanium, zirconium, and aluminum.

More preferably, the binder material is one or more of graphite, silica, titania, zirconia, alumina, and a mixed oxide of two or more of silicon, titanium, zirconium, and aluminum. More preferably, the binder material comprises silica, more preferably is silica.

In the case where the molding comprises a binder material, no particular restriction applies in view of the weight ratio of the zeolitic material relative to the binder material in the molding. It is preferred that, in the molding, the weight ratio of the zeolitic material relative to the binder material is in the range of from 1 to 20, preferably in the range of from 2 to 10, more preferably in the range of from 3 to 5.

As disclosed above, no particular restriction applies in view of the physical and/or chemical nature, e. g. the shape, of the molding provided that the molding comprises a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM. It is preferred that the molding has a rectangular, a triangular, a hexagonal, a square, an oval or a circular cross section. More preferably the molding is in the form of a star, a tablet, a sphere, a cylinder, a strand, or a hollow cylinder. More preferably, the molding has a rectangular, a triangular, a hexagonal, a square, an oval or a circular cross section and is in the form of a star, a tablet, a sphere, a cylinder, a strand, or a hollow cylinder.

As regards the one or more alkaline earth metals AEM further comprised in the zeolitic material, no particular restriction applies. It is preferred that the one or more alkaline earth metals AEM is one or more of Be, Mg, Ca, Sr and Ba, wherein the one or more alkaline earth metals AEM preferably comprises, more preferably is Mg.

As regards the physical and/or chemical nature of the one or more alkaline earth metals AEM in the zeolitic material, no particular restriction applies. It is preferred that the one or more alkaline earth metals AEM is present in the zeolitic material at least partly in an oxidic form. More preferably, the one or more alkaline earth metals AEM is present in the zeolitic material in an oxidic form.

As regards the amount of the one or more alkali metals AM, calculated as the element, comprised in the zeolitic material having an AEI-type framework structure, no particular restriction applies. It is preferred that the one or more alkali metals AM are comprised in the zeolitic material having an AEI-type framework structure in a total amount in the range of from 0.01 to 7 wt. % calculated as the element and based on 100 wt.-% of the total amount of Si, calculated as SiO₂, contained in the zeolitic material, preferably from 0.05 to 5 wt.-%, more preferably from 0.1 to 4 wt.-%, more preferably from 0.5 to 3.8 wt.-%, more preferably from 1 to 3.6 wt.-%, more preferably from 1.5 to 3.4 wt.-%, more preferably from 2 to 3.2 wt.-%, more preferably from 2.3 to 3 wt.-%, and more preferably from 2.5 to 2.9 wt.-%. According to the present invention it is particularly preferred that the one or more alkali metals AM are comprised in the zeolitic material having an AEI-type framework structure in a total amount in the range of from 2.6 to 2.8 wt.-%.

As regards the amount of the one or more alkaline earth metals AEM, calculated as the element, comprised in the zeolitic material, no particular restriction applies. It is preferred that the zeolitic material comprises the one or more alkaline earth metals AEM, calculated as the element, in a total amount in the range of from 0.1 to 5 weight-%, more preferably in the range of from 0.4 to 3 weight-%, more preferably in the range of from 0.75 to 2 weight-%, based on the weight of the zeolitic material comprised in the molding.

Therefore, it is particularly preferred that the zeolitic material comprises the one or more alkaline earth metals AEM, calculated as the element, in a total amount in the range of from 0.1 to 5 weight-%, more preferably in the range of from 0.4 to 3 weight-%, more preferably in the range of from 0.75 to 2 weight-%, based on the weight of the zeolitic material comprised in the molding, wherein the one or more alkaline earth metals AEM is one or more of Be, Mg, Ca, Sr and Ba, wherein the one or more alkaline earth metals AEM preferably comprises, more preferably is Mg, and wherein the one or more alkaline earth metals AEM is present in the zeolitic material at least partly in an oxidic form, more preferably, the one or more alkaline earth metals AEM is present in the zeolitic material in an oxidic form.

As disclosed above, no particular restriction applies in view of the physical and/or chemical nature of the molding. It is preferred that the molding comprises micropores having a diameter of less than 2 nanometer determined according to DIN 66135. Further, it is preferred that the molding comprises mesopores having a diameter in the range of from 2 to 50 nanometer determined according to DIN 66133. More preferably, the molding comprises micropores having a diameter of less than 2 nanometer determined according to DIN 66135 and mesopores having a diameter in the range of from 2 to 50 nanometer determined according to DIN 66133.

As disclosed above, no particular restriction applies in view of the physical and/or chemical nature of the molding. It is preferred that the molding comprised in the composition is a calcined molding, preferably calcined at a temperature in the range of from 400 to 600° C.

Further, the present invention relates to a process for preparing a molding, the process comprising

(i.1) providing a zeolitic material having an AEI-type framework structure, wherein the zeolitic material has a framework structure comprising Si, a trivalent element X, and oxygen;
(i.2) optionally impregnating the zeolitic material obtained from (i.1) with a source of the one or more alkaline earth metals AEM;
(i.3) preparing a molding comprising the impregnated zeolitic material obtained from (i.2) and optionally a binder material;
wherein the process is preferably a process as disclosed herein.

As disclosed above, no particular restriction applies in view of the physical and/or chemical nature of the molding. It is preferred that the molding comprised in the composition according to

(a) is obtainable or obtained by a process comprising
(i.1) providing a zeolitic material having an AEI-type framework structure, wherein the zeolitic material has a framework structure comprising Si, a trivalent element X, and oxygen;
(i.2) optionally impregnating the zeolitic material obtained from (i.1) with a source of the one or more alkaline earth metals AEM;
(i.3) preparing a molding comprising the impregnated zeolitic material obtained from (i.2) and optionally a binder material;
wherein the process is preferably a process as disclosed herein.

As disclosed above, the molding comprises a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM. Thus, no particular restriction applies in view of the element X. It is preferred that X is selected from the group consisting of Al, B, In, Ga, and mixtures of two or more thereof, X preferably being Al and/or B, and more preferably being Al.

As disclosed above, no particular restriction applies in view the physical and/or chemical nature of the molding comprised in the inventive composition. It is preferred that at least 95 weight-%, more preferably at least 98 weight-%, more preferably at least 99 weight-%, more preferably at least 99.5 weight-%, more preferably at least 99.9 weight % of the molding consist of the zeolitic material and optionally the binder material as disclosed herein.

As regards the physical and/or chemical nature of the mixed metal oxide, no particular restriction applies provided that the mixed metal oxide comprises chromium, zinc, and aluminum. It is preferred that at least 98 weight-%, preferably at least 99 weight-%, more preferably at least 99.5 weight-% of the mixed metal oxide consists of chromium, zinc, aluminum, and oxygen.

As disclosed above, no particular restriction applies in view of the physical and/or chemical nature, e. g. the BET specific surface area, of the mixed metal oxide provided that the mixed metal oxide comprises chromium, zinc, and aluminum. It is preferred that the mixed metal oxide has a BET specific surface area as determined according to DIN 66131 in the range of from 5 to 150 m²/g, more preferably in the range of from 15 to 120 m²/g.

In the case where at least 98 weight-%, preferably at least 99 weight-%, more preferably at least 99.5 weight-% of the mixed metal oxide consists of chromium, zinc, aluminum, and oxygen, as disclosed above, no particular restriction applies in view of the weight ratio of the zinc, calculated as element, relative to the chromium, calculated as element. It is preferred that, in the mixed metal oxide, the weight ratio of the zinc, calculated as element, relative to the chromium, calculated as element, is in the range of from 0.5 to 4, more preferably in the range of from 1 to 3.5, more preferably in the range of from 1.5 to 3, more preferably in the range of from 1.8 to 2.7, more preferably in the range of from 2 to 2.5, more preferably in the range of from 2.1 to 2.3, and more preferably in the range of from 2.15 to 2.25.

In the case where the mixed metal oxide has a BET specific surface area as determined according to DIN 66131 in the range of from 5 to 150 m²/g, more preferably in the range of from 15 to 120 m²/g, as disclosed above, again no particular restriction applies in view of the weight ratio of the zinc, calculated as element, relative to the chromium, calculated as element. It is preferred that, in the mixed metal oxide, the weight ratio of the zinc, calculated as element, relative to the chromium, calculated as element, is in the range of from 0.5 to 4, more preferably in the range of from 1 to 3.5, more preferably in the range of from 1.5 to 3, more preferably in the range of from 1.8 to 2.7, more preferably in the range of from 2 to 2.5, more preferably in the range of from 2.1 to 2.3, and more preferably in the range of from 2.15 to 2.25.

Therefore, it is particularly preferred that in the case where at least 98 weight-%, preferably at least 99 weight-%, more preferably at least 99.5 weight-% of the mixed metal oxide consists of chromium, zinc, aluminum, and oxygen, as disclosed above, and where the mixed metal oxide has a BET specific surface area as determined according to DIN 66131 in the range of from 5 to 150 m²/g, more preferably in the range of from 15 to 120 m²/g, as disclosed above, the weight ratio of the zinc, calculated as element, relative to the chromium, calculated as element, is in the range of from 0.5 to 4, more preferably in the range of from 1 to 3.5, more preferably in the range of from 1.5 to 3, more preferably in the range of from 1.8 to 2.7, more preferably in the range of from 2 to 2.5, more preferably in the range of from 2.1 to 2.3, and more preferably in the range of from 2.15 to 2.25, in the mixed metal oxide.

In the case where at least 98 weight-%, preferably at least 99 weight-%, more preferably at least 99.5 weight-% of the mixed metal oxide consists of chromium, zinc, aluminum, and oxygen, as disclosed above, or where the mixed metal oxide has a BET specific surface area as determined according to DIN 66131 in the range of from 5 to 150 m²/g, more preferably in the range of from 15 to 120 m²/g, as disclosed above, no particular restriction applies in view of the weight ratio of the aluminum, calculated as element, relative to the chromium, calculated as element. It is preferred that in the mixed metal oxide, the weight ratio of the aluminum, calculated as element, relative to the chromium, calculated as element, is in the range of from 0.5 to 3.5, more preferably in the range of from 1 to 3, more preferably in the range of from 1.5 to 2.7, more preferably in the range of from 1.8 to 2.5, more preferably in the range of from 2 to 2.25, and more preferably in the range of from 2.1 to 2.15.

Further, in the case where the weight ratio of the zinc, calculated as element, relative to the chromium, calculated as element, is in the range of from 0.5 to 4, preferably in the range of from 1 to 3.5, more preferably in the range of from 1.5 to 3, more preferably in the range of from 1.8 to 2.7, more preferably in the range of from 2 to 2.5, more preferably in the range of from 2.1 to 2.3, and more preferably in the range of from 2.15 to 2.25, in the mixed metal oxide, as disclosed above, no particular restriction applies in view of the weight ratio of the aluminum, calculated as element, relative to the chromium, calculated as element. It is preferred that in the mixed metal oxide, the weight ratio of the aluminum, calculated as element, relative to the chromium, calculated as element, is in the range of from 0.5 to 3.5, more preferably in the range of from 1 to 3, more preferably in the range of from 1.5 to 2.7, more preferably in the range of from 1.8 to 2.5, more preferably in the range of from 2 to 2.25, and more preferably in the range of from 2.1 to 2.15.

As disclosed above, the composition of the present invention contains a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM and a mixed metal oxide comprising chromium, zinc, and aluminum. Thus, no particular restriction applies in view of the weight ratio of the mixed metal oxide relative to the zeolitic material. According to a first alternative, it is preferred that the weight ratio of the mixed metal oxide relative to the zeolitic material is at least 0.2, more preferably in the range of from 0.2 to 5, more preferably in the range of from 0.5 to 3, more preferably in the range of from 0.9 to 1.5.

As disclosed above, the composition of the present invention contains a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM and a mixed metal oxide comprising chromium, zinc, and aluminum. Thus, no particular restriction applies in view of the weight ratio of the mixed metal oxide relative to the zeolitic material. According to a second alternative, it is preferred that the weight ratio of the mixed metal oxide relative to the zeolitic material is 0.2 or less, more preferably in the range of from 0.001 to 0.16, more preferably in the range of from 0.005 to 0.14, more preferably in the range of from 0.01 to 0.12, more preferably in the range of from 0.02 to 0.1, more preferably in the range of from 0.03 to 0.08, and more preferably in the range of from 0.04 to 0.06.

As regards the physical and/or chemical nature of the composition comprising a molding and a mixed metal oxide, as disclosed above, no particular restriction applies. It is preferred that at least 95 weight-%, more preferably at least 98 weight-%, more preferably at least 99 weight-%, more preferably at least 99.5 weight-%, more preferably at least 99.9 weight % of the composition consist of the molding and the mixed metal oxide.

As disclosed above, no particular restriction applies in view of the physical and/or chemical nature of the composition comprising a molding and a mixed metal oxide, as disclosed above. It is preferred that the composition is a mixture of the molding and the mixed metal oxide.

Further, the present invention relates to a process for preparing the composition as disclosed herein, the process comprising

(i) providing a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM;
(ii) providing a mixed metal oxide comprising chromium, zinc, and aluminum;
(iii) mixing the molding provided according to (i) with the mixed metal oxide provided according to (ii), obtaining the composition.

As regards the provision of a molding according to (i), no particular restriction applies such that providing a molding according to (i) may comprise further steps. It is preferred that providing a molding according to (i) comprises

(i.1) providing a zeolitic material having an AEI-type framework structure, wherein the zeolitic material has a framework structure comprising Si, a trivalent element X, and oxygen;
(i.2) optionally impregnating the zeolitic material obtained from (i.1) with a source of the one or more alkaline earth metals AEM;
(i.3) preparing a molding comprising the impregnated zeolitic material obtained from (i.2) or the zeolitic material from (i.1) and optionally a binder material.

As regards the trivalent element X comprised in the framework structure of the zeolitic material having an AEI-type framework structure according to (i.1), no particular restriction applies. It is preferred that X is selected from the group consisting of Al, B, In, Ga, and mixtures of two or more thereof, X preferably being Al and/or B, and more preferably being Al.

As regards the one or more alkaline earth metals AEM further comprised in the zeolitic material having an AEI-type framework structure according to (i.1), no particular restriction applies. It is preferred that the one or more alkaline earth metals AEM is one or more of Be, Mg, Ca, Sr and Ba, wherein the one or more alkaline earth metals AEM more preferably comprises, more preferably is Mg.

As regards the molar ratio Si:X, calculated as SiO₂:X₂O₃, of the zeolitic material provided in (i.1) having a framework structure comprising Si, a trivalent element X, and oxygen, no particular restriction applies. It is preferred that in the framework structure of the zeolitic material provided according to (i.1), the molar ratio Si:X, calculated as SiO₂:X₂O₃, is in the range of from 4 to 300, and preferably in the range of from 6 to 150, more preferably from 8 to 100, more preferably from 10 to 50, more preferably from 11 to 30, more preferably from 12 to 20, and more preferably from 12.5 to 16. According to the present invention it is particularly preferred that the molar ratio Si:X calculated as SiO₂:X₂O₃ of the framework structure of the zeolitic material provided in (i.1) is in the range of from 13 to 14.

As regards the physical and/or chemical nature of the framework structure of the zeolitic material provided according to (i.1), no particular restriction applies provided that the zeolitic material has an AEI-type framework structure, wherein the zeolitic material has a framework structure comprising Si, a trivalent element X, and oxygen. It is preferred that at least 95 weight-%, more preferably at least 98 weight-%, more preferably at least 99 weight-%, more preferably at least 99.5 weight-%, more preferably at least 99.9 weight % of the framework structure of the zeolitic material provided according to (i.1) consist of Si, X, O, and H.

As disclosed above, no particular restriction applies in view of the physical and/or chemical nature of the framework structure of the zeolitic material provided according to (i.1) provided that the zeolitic material has an AEI-type framework structure, wherein the zeolitic material has a framework structure comprising Si, a trivalent element X, and oxygen. Thus, further components, e. g. phosphorous, may be comprised the framework structure of the zeolitic material provided according to (i.1). It is preferred that at most 1 weight-%, more preferably at most 0.1 weight-%, more preferably at most 0.01 weight-%, more preferably from to 0.001 weight-% of the framework structure of the zeolitic material provided according to (i.1) consist of phosphorous.

As disclosed above, no particular restriction applies in view of the physical and/or chemical nature of the framework structure of the zeolitic material provided according to (i.1) as long as the zeolitic material has an AEI-type framework structure, wherein the zeolitic material has a framework structure comprising Si, a trivalent element X, and oxygen. It is preferred that at least 95 weight-%, more preferably at least 98 weight-%, more preferably at least 99 weight-%, more preferably at least 99.5 weight-%, more preferably at least 99.9 weight-% of the zeolitic material provided according to (i.1) consist of Si, X, 0, H, and the one or more alkali metals AM and/or the one or more alkaline earth metals AEM.

As regards the one or more alkali metals AM further comprised in the zeolitic material having an AEI-type framework structure and having a framework structure comprising Si, a trivalent element X, and oxygen, no particular restriction applies. It is preferred that the one or more alkali metals AM is one or more of Li, Na, K, Rb, and Cs, wherein the one or more alkali metals AM more preferably comprises, more preferably is Na.

As regards the physical and/or chemical nature of the source of the one or more alkaline earth metals AEM according to optional (i.2), no particular restriction applies provided that the zeolitic material obtained from (i.1) can be impregnated with the source of the one or more alkaline earth metals AEM. It is preferred that the source of the one or more alkaline earth metals according to (i.2) is a salt of the one or more alkaline earth metals.

As disclosed above, no particular restriction applies in view of the physical and/or chemical nature of the source of the one or more alkaline earth metals AEM according to optional (i.2) provided that the zeolitic material obtained from (i.1) can be impregnated with the source of the one or more alkaline earth metals AEM. It is preferred that the source of the one or more alkaline earth metals AEM according to (i.2) is a salt of the one or more alkaline earth metals dissolved in one or more solvents, preferably dissolved in water.

As regards optional impregnation of the zeolitic material obtained from (i.1) with a source of the one or more alkaline earth metals AEM according to (i.2), no particular restriction applies such that impregnating the zoelitic material can be achieved by any suitable method. It is preferred that the optional impregnation of the zeolitic material according to (i.2) comprises one or more of wet-impregnating the zeolitic material and spray-impregnating the zeolitic material, preferably spray-impregnating the zeolitic material.

As regards the optional impregnation of the zeolitic material obtained from (i.1) with a source of the one or more alkaline earth metals AEM, no particular restriction applies such that impregnating the zeolitic material according to (i.2) may comprise further steps, e. g. calcining the zeolitic material. It is preferred that (i.2) further comprises calcining the zeolitic material obtained from impregnation, optionally after drying the zeolitic material obtained from impregnation. In the case where the zeolitic material obtained from impregnation is calcined, optionally after drying the zeolitic material obtained from impregnation, it is preferred that the calcining is carried out in a gas atmosphere having a temperature in the range of from 400 to 650° C., more preferably in the range of from 450 to 600° C., wherein the gas atmosphere is preferably nitrogen, oxygen, air, lean air, or a mixture of two or more thereof. In the case where drying is carried out prior to calcining, the drying is preferably carried out in a gas atmosphere having a temperature in the range of from 75 to 200° C., more preferably in the range of from 90 to 150° C., wherein the gas atmosphere is preferably nitrogen, oxygen, air, lean air, or a mixture of two or more thereof.

As regards the physical and/or chemical nature of the impregnated zeolitic material obtained from (i.2), no particular restriction applies such that further components may be comprised therein. It is preferred that at least 95 weight-%, preferably at least 98 weight-%, more preferably at least 99 weight-%, more preferably at least 99.5 weight-%, more preferably at least 99.9 weight-% of the impregnated zeolitic material obtained from (i.2) consist of Si, X, 0, H, and the one or more alkali metals AM and/or the one or more alkaline earth metals AEM.

As regards the amount of the one or more alkali metals AM comprised in the zeolitic material having an AEI-type framework structure, no particular restriction applies. It is preferred that the one or more alkali metals AM are comprised in the zeolitic material having an AEI-type framework structure in a total amount in the range of from 0.01 to 7 wt.-% calculated as the element and based on 100 wt.-% of the total amount of Si, calculated as SiO₂, contained in the zeolitic material, preferably from 0.05 to 5 wt.-%, more preferably from 0.1 to 4 wt.-%, more preferably from 0.5 to 3.8 wt.-%, more preferably from 1 to 3.6 wt.-%, more preferably from 1.5 to 3.4 wt. %, more preferably from 2 to 3.2 wt.-%, more preferably from 2.3 to 3 wt.-%, and more preferably from 2.5 to 2.9 wt.-%. According to the present invention it is particularly preferred that the one or more alkali metals AM are comprised in the zeolitic material having an AEI-type framework structure in a total amount in the range of from 2.6 to 2.8 wt.-%.

As regards the amount of the one or more alkaline earth metals AEM comprised in the zeolitic material having an AEI-type framework structure, no particular restriction applies. It is preferred that the zeolitic material comprises the one or more alkaline earth metals AEM, calculated as the element, in a total amount in the range of from 0.1 to 5 weight-%, more preferably in the range of from 0.4 to 3 weight-%, more preferably in the range of from 0.75 to 2 weight-%, based on the total weight of the zeolitic material.

As disclosed above, the provision of a molding according to (i) comprises (i.1), (i.2) and (i.3). As regards the preparation of the molding according to (i.3), no particular restriction applies such that further steps may be included therein. It is preferred that preparing a molding according to

(i.3) comprises
(i.3.1) preparing a mixture of the impregnated zeolitic material obtained from (i.2) and a source of a binder material;
(i.3.2) subjecting the mixture prepared according to (i.3.1) to shaping.

In the case where the preparation of the molding according to (i.3) comprises (i.3.1) and (i.3.2), as disclosed above, no particular restriction applies in view of the physical and/or chemical nature of the source of a binder material. It is preferred that the source of a binder material is one or more of a source of graphite, a source of silica, a source of titania, a source of zirconia, a source of alumina and a source of a mixed oxide of two or more of silicon, titanium, zirconium and aluminum, wherein the source of a binder material more preferably comprises, more preferably is a source of silica, wherein the source of silica preferably comprises one or more of a colloidal silica, a fumed silica, and a tetraalkoxysilane, more preferably comprises a colloidal silica.

In the case where the preparation of the molding according to (i.3) comprises (i.3.1) and (i.3.2), as disclosed above, no particular restriction applies in view of the physical and/or chemical nature of the mixture prepared according to (i.3.1) such that further components, e. g. a pasting agent, may be comprised therein. It is preferred that the mixture prepared according to (i.3.1) further comprises a pasting agent, wherein the pasting agent preferably comprises one or more of an organic polymer, an alcohol and water. In the case where the pasting agent comprises an organic polymer, it is preferred that the organic polymer is one or more of a carbohydrate, a polyacrylate, a polymethacrylate, a polyvinyl alcohol, a polyvinylpyrrolidone, a polyisobutene, a polytetrahydrofuran, and a polyethlyene oxide, wherein the carbohydrate is preferably one or more of cellulose and cellulose derivative, wherein the cellulose derivative is preferably a cellulose ether, more preferably a hydroxyethyl methylcellulose. In the case where the mixture prepared according to (i.3.1) further comprises a pasting agent, it is particularly preferred that the pasting agent comprises one or more of water and a carbohydrate.

In the case where the preparation of the molding according to (i.3) comprises (i.3.1) and (i.3.2), as disclosed above, no particular restriction applies in view of subjecting to shaping according to (i.3.2) such that subjecting to shaping may be achieved by any suitable method. It is preferred that subjecting to shaping according to (i.3.2) comprises subjecting the mixture prepared according to (i.3.1) to spray-drying, to spray-granulation, or to extrusion, preferably to extrusion.

In the case where the preparation of the molding according to (i.3) comprises (i.3.1) and (i.3.2), as disclosed above, no particular restriction applies such that further steps, e. g. (i.3.3) may be included therein. It is preferred that the preparation of the molding according to (i.3) comprises (i.3.1), (i.3.2) and further (i.3.3), wherein (i.3.3) comprises calcining the molding obtained from (i.3.2), optionally after drying, wherein the calcining is preferably carried out in a gas atmosphere having a temperature in the range of from 500 to 950° C., more preferably in the range of from 600 to 900° C., more preferably in the range of from 650 to 850° C., more preferably in the range of from 700 to 820° C., and more preferably in the range of from 750 to 800° C. in the case where the preparation of the molding according to (i.3) comprises (i.3.1), (i.3.2) and further (i.3.3), it is preferred that the calcining is carried out in a gas atmosphere, wherein the gas atmosphere is preferably nitrogen, oxygen, air, lean air, or a mixture of two or more thereof, wherein, if drying is carried out prior to calcining, the drying is preferably carried out in a gas atmosphere having a temperature in the range of from 75 to 200° C., preferably in the range of from 90 to 150° C., wherein the gas atmosphere is preferably nitrogen, oxygen, air, lean air, or a mixture of two or more thereof.

As disclosed above, the process for preparing the composition as disclosed herein comprises (i), (ii) and (iii). As regards the provision of the mixed metal oxide comprising chromium, zinc, and aluminum according to (ii), no particular restriction applies such that further steps may be comprised therein. It is preferred that providing the mixed metal oxide according to (ii) comprises

(ii.1) co-precipitating a precursor of the mixed metal oxide from sources of the chromium, the zinc, and the aluminum;
(ii.2) washing the precursor obtained from (ii.1);
(ii.3) drying the washed precursor obtained from (ii.2);
(ii.4) calcining the washed precursor obtained from (ii.3).

In the case where providing the mixed metal oxide according to (ii) comprises (ii.1), (ii.2), (ii.3) and (ii.4), no particular restriction applies in view of co-precipitating a precursor according to

(ii.1) such that the co-precipitating may comprise further steps. It is preferred that co-precipitating a precursor according to (ii.1) comprises
(ii.1.1) preparing a mixture comprising water and the sources of the zinc and the aluminum, wherein the sources of the zinc and the aluminum preferably comprises one or more of a zinc salt and an aluminum salt, wherein more preferably, the zinc salt is a zinc nitrate, preferably a Zn(II) nitrate, and the aluminum salt is an aluminum nitrate, preferably an aluminum(III) nitrate;
(ii.1.2) adding a precipitation agent to the mixture prepared according to (ii.1.1), wherein the precipitation agent preferably comprises an ammonium carbonate, more preferably an ammonium carbonate dissolved in water;
(ii.1.3) subjecting the mixture obtained from (ii.1.2) to heating to a temperature of the mixture in the range of from 50 to 90° C., preferably in the range of from 60 to 80° C., and keeping the mixture at this temperature for a period of time, wherein the period of time is preferably in the range of from 0.1 to 12 h, more preferably in the range of from 0.5 to 6 h;
(ii.1.4) optionally drying the mixture obtained from (ii.1.3), preferably in a gas atmosphere having a temperature in the range of from 75 to 200° C., preferably in the range of from 90 to 150° C., wherein the gas atmosphere is preferably oxygen, air, lean air, or a mixture of two or more thereof;
(ii.1.5) calcining the mixture obtained from (ii.1.3) or from (ii.1.4), preferably from (ii.1.4), preferably in a gas atmosphere having a temperature in the range of from 300 to 900° C., preferably in the range of from 350 to 800° C., wherein the gas atmosphere is preferably oxygen, air, lean air, or a mixture of two or more thereof, obtaining the mixed metal oxide;
(ii.1.6) preparing a mixture comprising water and the source of the chromium, wherein the source of the chromium preferably comprises a chromium salt, wherein more preferably, the chromium salt is a chromium nitrate, preferably a chromium(III) nitrate;
(ii.1.7) impregnating the calcined mixed metal oxide obtained from (ii.1.5) with the mixture obtained from (ii.1.6), preferably by incipient wetness;
(ii.1.8) optionally drying the mixture obtained from (ii.1.7), preferably in a gas atmosphere having a temperature in the range of from 75 to 200° C., preferably in the range of from 90 to 150° C., wherein the gas atmosphere is preferably oxygen, air, lean air, ora mixture of two or more thereof;
(ii.1.9) calcining the mixture obtained from (ii.1.7) or from (ii.1.8), preferably from (ii.1.8), preferably in a gas atmosphere having a temperature in the range of from 300 to 900° C., preferably in the range of from 350 to 800° C., wherein the gas atmosphere is preferably oxygen, air, lean air, or a mixture of two or more thereof, obtaining the impregnated mixed metal oxide.

In the case where the co-precipitating a precursor according to (ii.1) comprises (ii.1.1), (ii.1.2), (ii.1.3), optionally (ii.1.4), (ii.1.5), (ii.1.6), (ii.1.7), optionally (ii.1.8) and (ii.1.9), no particular restriction applies in view of the conditions, e. g. the temperature, at which the mixture is calcined according to (ii.5) and/or (ii.9). According to a first alternative, the mixture is calcined according to (ii.1.5) and/or (ii.1.9), preferably according to (ii.1.5) and (ii.1.9), at a temperature in the range of from 350 to 440° C., preferably in the range of from 375 to 425° C. According to a second alternative, the mixture is calcined according to (ii.1.5) and/or (ii.1.9), preferably according to (ii.1.5) and (ii.1.9), at a temperature in the range of from 450 to 550° C., preferably in the range of from 475 to 525° C. According to a third alternative, the mixture is calcined according to (ii.1.5) and/or (ii.1.9), preferably according to (ii.1.5) and (ii.1.9), at a temperature in the range of from 700 to 800° C., preferably in the range of from 725 to 775° C.

In the case where the co-precipitating a precursor according to (ii.1) comprises (ii.1.1), (ii.1.2), (ii.1.3), optionally (ii.1.4), (ii.1.5), (ii.1.6), (ii.1.7), optionally (ii.1.8) and (ii.1.9), no particular restriction applies in view of the weight ratio of the zinc, calculated as element, relative to the aluminum, calculated as element, in the mixture prepared in (ii.1.1). It is preferred that in the mixture prepared in (ii.1.1), the weight ratio of the zinc, calculated as element, relative to the aluminum, calculated as element, is in the range of from 0.5 to 2, more preferably in the range of from 0.8 to 1.7, more preferably in the range of from 0.9 to 1.5, more preferably in the range of from 1 to 1.25, and more preferably in the range of from 1.1 to 1.15.

Further, in the case where the co-precipitating a precursor according to (ii.1) comprises (ii.1.1), (ii.1.2), (ii.1.3), optionally (ii.1.4), (ii.1.5), (ii.1.6), (ii.1.7), optionally (ii.1.8) and (ii.1.9), no particular restriction applies in view of the weight ratio of the aluminum, calculated as element, relative to the chromium, calculated as element, in the mixture prepared in (ii.1.7). It is preferred that the weight ratio of the aluminum, calculated as element, relative to the chromium, calculated as element, in the mixture prepared in (ii.1.7) is in the range of from 1.5 to 4, more preferably in the range of from 2 to 3.5, more preferably in the range of from 2.3 to 3.2, and more preferably in the range of from 2.5 to 3.

Therefore, it is particularly preferred that, in the case where the co-precipitating a precursor according to (ii.1) comprises (ii.1.1), (ii.1.2), (ii.1.3), optionally (ii.1.4), (ii.1.5), (ii.1.6), (ii.1.7), optionally (ii.1.8) and (ii.1.9), the weight ratio of the zinc, calculated as element, relative to the aluminum, calculated as element, is in the range of from 0.5 to 2, more preferably in the range of from 0.8 to 1.7, more preferably in the range of from 0.9 to 1.5, more preferably in the range of from 1 to 1.25, and more preferably in the range of from 1.1 to 1.15, and that the weight ratio of the aluminum, calculated as element, relative to the chromium, calculated as element, in the mixture prepared in (ii.1.7) is in the range of from 1.5 to 4, more preferably in the range of from 2 to 3.5, more preferably in the range of from 2.3 to 3.2, and more preferably in the range of from 2.5 to 3.

Further, the present invention relates to a molding, obtainable or obtained by a process as disclosed herein.

Furthermore, the present invention relates to a mixed metal oxide, obtainable or obtained by a process as disclosed herein.

Moreover, the present invention relates to a composition, obtainable or obtained by a process as disclosed herein.

In addition, the present invention relates to a use of a composition as disclosed herein as a catalyst or as a catalyst component, preferably for preparing C2 to C4 olefins, more preferably for preparing C2 to C4 olefins from a synthesis gas comprising hydrogen and carbon monoxide, wherein the C2 to C4 olefins is preferably one or more of ethene and propene, more preferably propene, wherein preparing the C2 to C4 olefins is preferably carried out as a one-step process.

Further, the present invention relates to a process for preparing C2 to C4 olefins from a synthesis gas comprising hydrogen and carbon monoxide, the process comprising

(1) providing a gas stream which comprises a synthesis gas stream comprising hydrogen and carbon monoxide;
(2) providing a catalyst comprising a composition as disclosed herein;
(3) bringing the gas stream provided in (1) in contact with the catalyst provided in (2), obtaining a reaction mixture stream comprising C2 to C4 olefins.

As regards the molar ratio of hydrogen relative to carbon monoxide in the synthesis gas stream provided in (1), no particular restriction applies. It is preferred that the molar ratio of hydrogen relative to carbon monoxide in the synthesis gas stream provided in (1) is in the range of from 0.1 to 10, more preferably in the range of from 0.2 to 5, more preferably in the range of from 0.25 to 2.

As regards the physical and/or chemical nature of the synthesis gas stream according to (1), no particular restriction applies such that further components may be comprised therein. It is preferred that at least 99 volume-%, more preferably at least 99.5 volume-%, more preferably at least 99.9 volume-% of the synthesis gas stream according to (1) consist of hydrogen and carbon monoxide.

Therefore, it is particularly preferred that the molar ratio of hydrogen relative to carbon monoxide in the synthesis gas stream provided in (1) is in the range of from 0.1 to 10, more preferably in the range of from 0.2 to 5, more preferably in the range of from 0.25 to 2, and that preferably at least 99 volume-%, more preferably at least 99.5 volume-%, more preferably at least 99.9 volume-% of the synthesis gas stream according to (1) consist of hydrogen and carbon monoxide.

As regards the physical and/or chemical nature of the gas stream provided in (1), no particular restriction applies such that further components may be comprised therein. It is preferred that at least 80 volume-%, more preferably at least 85 volume-%, more preferably at least 90 volume-%, more preferably from 90 to 99 volume-% of the gas stream provided in (1) consist of the synthesis gas stream.

As disclosed above, no particular restriction applies in view of the physical and/or chemical nature of the gas stream provided in (1) such that further components may be comprised therein. It is preferred that the gas stream provided in (1) further comprises one or more inert gas preferably comprising, more preferably being one or more of nitrogen and argon.

In the case where the gas stream provided in (1) further comprises one or more inert gas, no particular restriction applies in view of the volume ratio of the one or more inter gases relative to the synthesis gas stream. It is preferred that the volume ratio of the one or more inter gases relative to the synthesis gas stream in the gas stream provided in (1) is in the range of from 1:20 to 1:2, preferably in the range of from 1:15 to 1:5, more preferably in the range of from 1:12 to 1:8.

Further, in the case where the gas stream provided in (1) further comprises one or more inert gas, no particular restriction applies in view of the physical and/or chemical nature of the gas stream provided in (1) such that further components may be comprised therein. It is preferred that at least 99 volume-%, more preferably at least 99.5 volume-%, more preferably at least 99.9 volume-% of the gas stream provided in (1) consist of the synthesis gas stream and the one or more inert gases.

Therefore, it is particularly preferred that the gas stream provided in (1) further comprises one or more inert gas preferably comprising, more preferably being one or more of nitrogen and argon, that the volume ratio of the one or more inter gases relative to the synthesis gas stream in the gas stream provided in (1) is preferably in the range of from 1:20 to 1:2, preferably in the range of from 1:15 to 1:5, more preferably in the range of from 1:12 to 1:8, and that preferably at least 99 volume-%, more preferably at least 99.5 volume-%, more preferably at least 99.9 volume-% of the gas stream provided in (1) consist of the synthesis gas stream and the one or more inert gases.

As disclosed above, the process for preparing C2 to C4 olefins from a synthesis gas comprising hydrogen and carbon monoxide comprises (1), (2) and (3). As regards bringing the gas stream provided in (1) in contact with the catalyst provided in (2) according to (3), no particular restriction applies in view of the conditions, e. g. the temperature, under which the gas stream provided in (1) is brought, according to (3), in contact with the catalyst provided in (2), provided that a reaction mixture stream comprising C2 to C4 olefins can be obtained. It is preferred that, according to (3), the gas stream is brought in contact with the catalyst at a temperature of the gas stream in the range of from 200 to 550° C., more preferably in the range of from 250 to 525° C., more preferably in the range of from 300 to 500° C.

Further, as disclosed above, the process for preparing C2 to C4 olefins from a synthesis gas comprising hydrogen and carbon monoxide comprises (1), (2) and (3). As regards bringing the gas stream provided in (1) in contact with the catalyst provided in (2) according to (3), no particular restriction applies in view of the conditions, e. g. the pressure, under which the gas stream provided in (1) is brought, according to (3), in contact with the catalyst provided in (2), provided that a reaction mixture stream comprising C2 to C4 olefins can be obtained. It is preferred that, according to (3), the gas stream is brought in contact with the catalyst at a pressure of the gas stream in the range of from 10 to 40 bar(abs), more preferably in the range of from 12.5 to 30 bar(abs), more preferably in the range of from 15 to 25 bar(abs).

Therefore, it is particularly preferred that, according to (3), the gas stream is brought in contact with the catalyst at a temperature of the gas stream in the range of from 200 to 550° C., more preferably in the range of from 250 to 525° C., more preferably in the range of from 300 to 500° C., and preferably at a pressure of the gas stream in the range of from 10 to 40 bar(abs), more preferably in the range of from 12.5 to 30 bar(abs), more preferably in the range of from 15 to 25 bar(abs).

As disclosed above, the process for preparing C2 to C4 olefins from a synthesis gas comprising hydrogen and carbon monoxide comprises (1), (2) and (3). As regards the manner in which according to (2) the catalyst comprising a composition as disclosed herein is provided, no particular restriction applies. It is preferred that the catalyst provided in (2) is comprised in a reactor tube, the reactor tube preferably comprising a catalyst bed, and the catalyst bed preferably comprising the catalyst provided in (2), and wherein bringing the gas stream provided in (1) in contact with the catalyst provided in (2) according to (3) comprises passing the gas stream as feed stream into the reactor tube and through the catalyst bed comprised in the reactor tube, whereby the reaction mixture stream can be obtained comprising C2 to C4 olefins, said process preferably further comprises removing the reaction mixture stream from the reactor tube.

In the case where the catalyst provided in (2) is comprised in a reactor tube, the reactor tube preferably comprising a catalyst bed, and the catalyst bed preferably comprising the catalyst provided in (2), and wherein bringing the gas stream provided in (1) in contact with the catalyst provided in (2) according to (3) comprises passing the gas stream as feed stream into the reactor tube and through the catalyst bed comprised in the reactor tube, whereby the reaction mixture stream can be obtained comprising C2 to C4 olefins, said process preferably further comprises removing the reaction mixture stream from the reactor tube, no particular restriction applies in view of the physical and/or chemical nature, e. g. the gas hourly space velocity, of the gas stream. It is preferred that, according to (3), the gas stream is brought in contact with the catalyst at a gas hourly space velocity in the range of from 100 to 25,000 h⁻¹, more preferably in the range of from 500 to 20,000 h⁻¹, more preferably in the range of from 1,000 to 10,000 h⁻¹, wherein the gas hourly space velocity is defined as the volume flow rate of the gas stream brought in contact with the catalyst divided by the volume of the catalyst bed.

As disclosed above, the process for preparing C2 to C4 olefins from a synthesis gas comprising hydrogen and carbon monoxide comprises (1), (2) and (3). As regards the physical and/or chemical nature of the catalyst provided in (2), no particular restriction applies. It is preferred that the catalyst provided in (2) is activated prior to (3).

In the case where the catalyst is activated, no particular restriction applies in view of the method of activation such that any method for activating the catalyst may be applied. It is preferred that activating the catalyst comprises bringing the catalyst in contact with a gas stream comprising hydrogen and an inert gas, wherein preferably from 1 to 50 volume-%, more preferably from 2 to 35 volume-%, more preferably from 5 to 20 volume-% of the gas stream consist of hydrogen, and wherein the inert gas preferably comprises one or more of nitrogen and argon, more preferably nitrogen.

In the case where activating the catalyst comprises bringing the catalyst in contact with a gas stream comprising hydrogen and an inert gas as disclosed above, no particular restriction applies in view of the gas stream comprising hydrogen such that further components may be comprised therein. It is preferred that at least 98 volume-%, preferably at least 99 volume-%, more preferably at least 99.5 volume-% of the gas stream comprising hydrogen consist of hydrogen and the inert gas.

In the case where activating the catalyst comprises bringing the catalyst in contact with a gas stream comprising hydrogen and an inert gas as disclosed above, no particular restriction applies in view of the conditions, e. g. the temperature, under which the gas stream comprising hydrogen is brought in contact with the catalyst. It is preferred that the gas stream comprising hydrogen is brought in contact with the catalyst at a temperature of the gas stream in the range of from 200 to 400° C., more preferably in the range of from 250 to 350° C., more preferably in the range of from 275 to 325° C.

In the case where activating the catalyst comprises bringing the catalyst in contact with a gas stream comprising hydrogen and an inert gas as disclosed above, no particular restriction applies in view of the conditions, e. g. the pressure, under which the gas stream comprising hydrogen is brought in contact with the catalyst. It is preferred that the gas stream comprising hydrogen is brought in contact with the catalyst at a pressure of the gas stream in the range of from 1 to 50 bar(abs), preferably in the range of from 5 to 40 bar(abs), more preferably in the range of from 10 to 30 bar(abs).

Further, in the case where activating the catalyst comprises bringing the catalyst in contact with a gas stream comprising hydrogen and an inert gas as disclosed above, no particular restriction applies in view of the manner in which the catalyst is provided in (2). It is preferred that the catalyst provided in (2) is comprised in a reactor tube, the reactor tube preferably comprising a catalyst, the catalyst bed preferably comprising the catalyst provided in (2), and wherein prior to (3), bringing the gas stream comprising hydrogen in contact with the catalyst provided in (2) comprises passing the gas stream comprising hydrogen into the reactor tube and through the catalyst bed comprised in the reactor tube.

In the case where the catalyst provided in (2) is comprised in a reactor tube, the reactor tube preferably comprising a catalyst, the catalyst bed preferably comprising the catalyst provided in (2), and wherein prior to (3), bringing the gas stream comprising hydrogen in contact with the catalyst provided in (2) comprises passing the gas stream comprising hydrogen into the reactor tube and through the catalyst bed comprised in the reactor tube, as disclosed above, no particular restriction applies in view of the conditions, e. g. the gas hourly space velocity, under which the gas stream comprising hydrogen is brought in contact with the catalyst. It is preferred that the gas stream comprising hydrogen is brought in contact with the catalyst at a gas hourly space velocity in the range of from 500 to 15,000 h⁻¹, more preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed.

As disclosed above, the process for preparing C2 to C4 olefins from a synthesis gas comprising hydrogen and carbon monoxide comprises (1), (2) and (3). Further, as disclosed above, the catalyst provided in (2) is preferably activated prior to (3). In the case where the catalyst provided in (2) is activated prior to (3), no particular restriction applies in view of the method with which activating the catalyst is achieved such that any suitable activation method may be applied. It is preferred that activating the catalyst further comprises bringing the catalyst in contact with a synthesis gas stream comprising hydrogen and carbon monoxide, wherein in the synthesis gas stream the molar ratio of hydrogen relative to carbon monoxide is preferably in the range of from 0.1 to 10, more preferably in the range of from 0.2 to 5, more preferably in the range of from 0.25 to 2, wherein preferably at least 99 volume-%, more preferably at least 99.5 volume-%, more preferably at least 99.9 volume-% of the synthesis gas stream according to (1) consist of hydrogen and carbon monoxide.

In the case where activating the catalyst further comprises bringing the catalyst in contact with a synthesis gas stream comprising hydrogen and carbon monoxide, as disclosed above, no particular restriction applies in view of the physical and/or chemical nature of the synthesis gas stream comprising hydrogen and carbon monoxide used for activating the catalyst. It is preferred that the synthesis gas stream comprising hydrogen and carbon monoxide used for activating the catalyst is the synthesis gas stream provided in (1).

Further, in the case where activating the catalyst further comprises bringing the catalyst in contact with a synthesis gas stream comprising hydrogen and carbon monoxide as disclosed above, no particular restriction applies in view of the conditions, e. g. the temperature, under which the synthesis gas stream comprising hydrogen and carbon monoxide is brought in contact with the catalyst for activating the catalyst. It is preferred that, for activating the catalyst, the synthesis gas stream comprising hydrogen and carbon monoxide is brought in contact with the catalyst at a temperature of the gas stream in the range of from 100 to 300° C., more preferably in the range of from 150 to 275° C., more preferably in the range of from 200 to 250° C.

Further, in the case where activating the catalyst further comprises bringing the catalyst in contact with a synthesis gas stream comprising hydrogen and carbon monoxide as disclosed above, no particular restriction applies in view of the conditions, e. g. the pressure, under which the synthesis gas stream comprising hydrogen and carbon monoxide is brought in contact with the catalyst for activating the catalyst. It is preferred that, for activating the catalyst, the synthesis gas stream comprising hydrogen and carbon monoxide is brought in contact with the catalyst at a pressure of the gas stream in the range of from 10 to 50 bar(abs), more preferably in the range of from 15 to 35 bar(abs), more preferably in the range of from 20 to 30 bar(abs).

In the case where activating the catalyst further comprises bringing the catalyst in contact with a synthesis gas stream comprising hydrogen and carbon monoxide, as disclosed above, no particular restriction applies in view of the manner in which the catalyst is provided in (2). It is preferred that the catalyst provided in (2) is comprised in a reactor tube, the reactor tube preferably comprising a catalyst, the catalyst bed preferably comprising the catalyst provided in (2), and wherein for activating the catalyst, bringing the synthesis gas stream comprising hydrogen and carbon monoxide in contact with the catalyst provided in (2) comprises passing the synthesis gas stream comprising hydrogen and carbon monoxide into the reactor tube and through the catalyst bed comprised in the reactor tube.

In the case where the catalyst provided in (2) is comprised in a reactor tube, the reactor tube preferably comprising a catalyst, the catalyst bed preferably comprising the catalyst provided in (2), and wherein for activating the catalyst, bringing the synthesis gas stream comprising hydrogen and carbon monoxide in contact with the catalyst provided in (2) comprises passing the synthesis gas stream comprising hydrogen and carbon monoxide into the reactor tube and through the catalyst bed comprised in the reactor tube, as disclosed above, no particular restriction applies in view of the conditions, e. g. the gas hourly space velocity, under which the synthesis gas stream comprising hydrogen and carbon monoxide is brought in contact with the catalyst. It is preferred that the synthesis gas stream comprising hydrogen and carbon monoxide is brought in contact with the catalyst at a gas hourly space velocity in the range of from 500 to 15,000 h⁻¹, more preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed.

Further, in the case where the catalyst provided in (2) is comprised in a reactor tube, the reactor tube preferably comprising a catalyst, the catalyst bed preferably comprising the catalyst provided in (2), and wherein for activating the catalyst, bringing the synthesis gas stream comprising hydrogen and carbon monoxide in contact with the catalyst provided in (2) comprises passing the synthesis gas stream comprising hydrogen and carbon monoxide into the reactor tube and through the catalyst bed comprised in the reactor tube, as disclosed above, no particular restriction applies in view of the order of process steps for activating the catalyst prior to (3). It is preferred that, for activating the catalyst prior to (3), bringing the synthesis gas stream comprising hydrogen and carbon monoxide in contact with the catalyst provided in (2) is carried out prior to bringing the catalyst in contact with a gas stream comprising hydrogen and an inert gas according to any one of the aforementioned particular and preferred embodiments.

As disclosed above, the process for preparing C2 to C4 olefins from a synthesis gas comprising hydrogen and carbon monoxide comprises (1), (2) and (3). As regards the C2 to C4 olefins, it is preferred that the C2 to C4 olefins comprise, preferably consist of ethene, propene, and a butene, wherein the butene is preferably 1-butene.

In the case where the C2 to C4 olefins comprise ethene, propene and a butene, preferably consist of ethene, propene and a butane, as disclosed above, no particular restriction applies in view of the molar ratio of propene relative to ethene and the molar ratio of ethene relative to the butene. It is preferred that the molar ratio of propene relative to ethene is greater than 1 and the molar ratio of ethene relative to the butene is greater than 1.

As disclosed above, the process for preparing C2 to C4 olefins from a synthesis gas comprising hydrogen and carbon monoxide comprises (1), (2) and (3). As regards the conversion of the synthesis gas to the C2 to C4 olefins, no particular restriction applies. It is preferred that the conversion of the synthesis gas to the C2 to C4 olefins exhibits a selectivity towards the C2 to C4 olefins of at least 30%. According to the present invention, it is preferred that the selectivity is determined as described in the experimental section.

The present invention is further illustrated by the following embodiments and combinations of embodiments as indicated by the respective dependencies and back-references. In particular, it is noted that in each instance where a combination of embodiments is mentioned as a range, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Thus, the present invention includes the following embodiments, wherein these include the specific combinations of embodiments as indicated by the respective interdependencies defined therein:
1. A composition comprising
   a) a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM; and
   b) a mixed metal oxide comprising chromium, zinc, and aluminum.
2. The composition of embodiment 1, wherein X is selected from the group consisting of Al, B, In, Ga, and mixtures of two or more thereof, X preferably being Al and/or B, and more preferably being Al.
3. The composition of embodiment 1 or 2, wherein the one or more alkali metals AM is one or more of Li, Na, K, Rb, and Cs, wherein the one or more alkali metals AM preferably comprises, more preferably is Na.
4. The composition of any one of embodiments 1 to 3, wherein in the framework structure of the zeolitic material, the molar ratio Si:X calculated as SiO₂:X₂O₃ is in the range of from 4 to 300, preferably from 6 to 150, more preferably from 8 to 100, more preferably from 10 to 50, more preferably from 11 to 30, more preferably from 12 to 20, more preferably from 12.5 to 16, and more preferably from 13 to 14.
5. The composition of any one of embodiments 1 to 4, wherein at least 95 weight-%, preferably at least 98 weight-%, more preferably at least 99 weight-%, more preferably at least 99.5 weight-%, more preferably at least 99.9 weight % of the framework structure of the zeolitic material consist of Si, X, O, and H.
6. The composition of any one of embodiments 1 to 5, wherein at most 1 weight-%, preferably at most 0.1 weight-%, more preferably at most 0.01 weight-%, more preferably from 0 to 0.001 weight-% of the framework structure of the zeolitic material consist of phosphorous.
7. The composition of any one of embodiments 1 to 6, wherein at least 95 weight-%, preferably at least 98 weight-%, more preferably at least 99 weight-%, more preferably at least 99.5 weight-%, more preferably at least 99.9 weight-% of the zeolitic material consist of Si, X, O, H, and the one or more alkali metals AM and/or the one or more alkaline earth metals AEM.
8. The composition of any one of embodiment 1 to 7, wherein the molding further comprises a binder material.
9. The composition of embodiment 8, wherein the binder material comprises, preferably is one or more of graphite, silica, titania, zirconia, alumina, and a mixed oxide of two or more of silicon, titanium, zirconium, and aluminum, wherein more preferably, the binder material comprises silica, more preferably is silica.
10. The composition of embodiment 8 or 9, wherein in the molding, the weight ratio of the zeolitic material relative to the binder material is in the range of from 1 to 20, preferably in the range of from 2 to 10, more preferably in the range of from 3 to 5.
11. The composition of any one of embodiments 1 to 10, wherein the molding has a rectangular, a triangular, a hexagonal, a square, an oval or a circular cross section, and/or preferably is in the form of a star, a tablet, a sphere, a cylinder, a strand, or a hollow cylinder.
12. The composition of any one of embodiments 1 to 11, wherein the one or more alkaline earth metals AEM is one or more of Be, Mg, Ca, Sr and Ba, wherein the one or more alkaline earth metals AEM preferably comprises, more preferably is Mg.
13. The composition of any one of embodiments 1 to 12, wherein the one or more alkaline earth metals AEM is present in the zeolitic material at least partly in an oxidic form.
14. The composition of any one of embodiments 1 to 13, wherein the one or more alkali metals AM are comprised in the zeolitic material having an AEI-type framework structure in a total amount in the range of from 0.01 to 7 wt.-% calculated as the element and based on 100 wt.-% of the total amount of Si, calculated as SiO₂, contained in the zeolitic material, preferably from 0.05 to 5 wt.-%, more preferably from 0.1 to 4 wt.-%, more preferably from 0.5 to 3.8 wt.-%, more preferably from 1 to 3.6 wt.-%, more preferably from 1.5 to 3.4 wt. %, more preferably from 2 to 3.2 wt.-%, more preferably from 2.3 to 3 wt.-%, more preferably from 2.5 to 2.9 wt.-%, and more preferably from 2.6 to 2.8 wt.-%.
15. The composition of any one of embodiments 1 to 14, wherein the zeolitic material comprises the one or more alkaline earth metals AEM, calculated as the element, in a total amount in the range of from 0.1 to 5 weight-%, preferably in the range of from 0.4 to 3 weight-%, more preferably in the range of from 0.75 to 2 weight-%, based on the weight of the zeolitic material comprised in the molding.
16. The composition of any one of embodiments 1 to 15, wherein the molding comprises micropores having a diameter of less than 2 nanometer determined according to DIN 66135 and comprises mesopores having a diameter in the range of from 2 to 50 nanometer determined according to DIN 66133.
17. The composition of any one of embodiments 1 to 16, wherein the molding comprised in the composition is a calcined molding, preferably calcined at a temperature in the range of from 400 to 600° C.
18. The composition of any one of embodiments 1 to 17, wherein the molding according to (a) is obtainable or obtained by a process comprising
   (i.1) providing a zeolitic material having an AEI-type framework structure, wherein the zeolitic material has a framework structure comprising Si, a trivalent element X, and oxygen;
   (i.2) optionally impregnating the zeolitic material obtained from (i.1) with a source of the one or more alkaline earth metals AEM;
   (i.3) preparing a molding comprising the impregnated zeolitic material obtained from (i.2) and optionally a binder material;
   wherein the process is preferably a process according to any one of embodiments 30 to 49.
19. The composition of any one of embodiments 1 to 18, wherein X is selected from the group consisting of Al, B, In, Ga, and mixtures of two or more thereof, X preferably being Al and/or B, and more preferably being Al.
20. The composition of any one of embodiments 1 to 19, wherein the zeolitic material having an AEI-type framework structure displays a total amount of acid sites as determined by NH₃-TPD in the range of from 0.1 to 3 mmol/g, preferably from 0.3 to 2.5 mmol/g, more preferably from 0.5 to 2.2 mmol/g, more preferably from 0.8 to 2 mmol/g, more preferably from 1 to 1.8 mmol/g, more preferably from 1.1 to 1.7 mmol/g, more preferably from 1.2 to 1.6 mmol/g, more preferably from 1.3 to 1.5 mmol/g, and more preferably from 1.35 to 1.45 mmol/g, wherein preferably the NH₃-TPD is obtained according to the method described in the experimental section of the present application.
21. The composition of any one of embodiments 1 to 20, wherein the zeolitic material having an AEI-type framework structure displays a NH₃-TPD desorption spectrum, preferably a deconvoluted NH₃-TPD desorption spectrum, comprising a peak in the range of from 400 to 600° C., preferably from 430 to 600° C., more preferably from 450 to 580° C., more preferably from 480 to 550° C., more preferably from 500 to 530° C., and more preferably from 510 to 520° C., wherein preferably the NH₃-TPD is obtained according to the method described in the experimental section of the present application.
22. The composition of embodiment 21, wherein the integration of the peak affords an amount of acid sites in the range of from 0.05 to 1.5 mmol/g, preferably from 0.1 to 1.2 mmol/g, more preferably from 0.2 to 1 mmol/g, more preferably from 0.3 to 0.9 mmol/g, more preferably from 0.4 to 0.8 mmol/g, more preferably from 0.5 to 0.7 mmol/g, and more preferably from 0.55 to 0.65 mmol/g.
23. The composition of any one of embodiments 1 to 22, wherein the molding comprising a zeolitic material having an AEI-type framework structure displays a total amount of acid sites as determined by NH₃-TPD in the range of from 0.05 to 1.8 mmol/g, preferably from 0.1 to 1.5 mmol/g, more preferably from 0.3 to 1.3 mmol/g, more preferably from 0.5 to 1.2 mmol/g, more preferably from 0.6 to 1.1 mmol/g, more preferably from 0.7 to 1 mmol/g, more preferably from 0.8 to 0.95 mmol/g, and more preferably from 0.85 to 0.9 mmol/g, wherein preferably the NH₃-TPD is obtained according to the method described in the experimental section of the present application.
24. The composition of any one of embodiments 1 to 23, wherein the molding comprising a zeolitic material having an AEI-type framework structure displays a NH₃-TPD desorption spectrum, preferably a deconvoluted NH₃-TPD desorption spectrum, comprising a peak in the range of from 300 to 550° C., preferably from 330 to 520° C., more preferably from 350 to 500° C., more preferably from 380 to 480° C., more preferably from 400 to 450° C., more preferably from 410 to 430° C., and more preferably from 415 to 420° C., wherein preferably the NH₃-TPD is obtained according to the method described in the experimental section of the present application.
25. The composition of embodiment 24, wherein the integration of the peak affords an amount of acid sites in the range of from 0.01 to 0.3 mmol/g, preferably from 0.02 to 0.2 mmol/g, more preferably from 0.03 to 0.15 mmol/g, more preferably from 0.04 to 0.12 mmol/g, more preferably from 0.05 to 0.1 mmol/g, and more preferably from 0.06 to 0.08 mmol/g.
26. The composition of any one of embodiments 1 to 25, wherein at least 95 weight-%, preferably at least 98 weight-%, more preferably at least 99 weight-%, more preferably at least 99.5 weight-%, more preferably at least 99.9 weight % of the molding consist of the zeolitic material and optionally the binder material according to any one of embodiments 9 to 11.
27. The composition of any one of embodiments 1 to 26, wherein at least 98 weight-%, preferably at least 99 weight-%, more preferably at least 99.5 weight-% of the mixed metal oxide consists of chromium, zinc, aluminum, and oxygen.
28. The composition of any one of embodiments 1 to 27, wherein the mixed metal oxide has a BET specific surface area as determined according to DIN 66131 in the range of from 5 to 150 m²/g, preferably in the range of from 15 to 120 m²/g.
29. The composition of embodiment 21 or 28, wherein in the mixed metal oxide, the weight ratio of the zinc, calculated as element, relative to the chromium, calculated as element, is in the range of from 0.5 to 4, preferably in the range of from 1 to 3.5, more preferably in the range of from 1.5 to 3, more preferably in the range of from 1.8 to 2.7, more preferably in the range of from 2 to 2.5, more preferably in the range of from 2.1 to 2.3, and more preferably in the range of from 2.15 to 2.25.
30. The composition of any one of embodiments 21 to 29, wherein in the mixed metal oxide, the weight ratio of the aluminum, calculated as element, relative to the chromium, calculated as element, is in the range of from 0.5 to 3.5, preferably in the range of from 1 to 3, more preferably in the range of from 1.5 to 2.7, more preferably in the range of from 1.8 to 2.5, more preferably in the range of from 2 to 2.25, and more preferably in the range of from 2.1 to 2.15.
31. The composition of any one of embodiments 1 to 30, wherein the weight ratio of the mixed metal oxide relative to the zeolitic material is at least 0.2, preferably in the range of from 0.2 to 5, more preferably in the range of from 0.5 to 3, more preferably in the range of from 0.9 to 1.5.
32. The composition of any one of embodiments 1 to 31, wherein the weight ratio of the mixed metal oxide relative to the zeolitic material is 0.2 or less, preferably in the range of from 0.001 to 0.16, more preferably in the range of from 0.005 to 0.14, more preferably in the range of from 0.01 to 0.12, more preferably in the range of from 0.02 to 0.1, more preferably in the range of from 0.03 to 0.08, and more preferably in the range of from 0.04 to 0.06.
33. The composition of any one of embodiments 1 to 32, wherein at least 95 weight-%, preferably at least 98 weight-%, more preferably at least 99 weight-%, more preferably at least 99.5 weight-%, more preferably at least 99.9 weight % of the composition consist of the molding and the mixed metal oxide.
34. The composition of any one of embodiments 1 to 33, wherein the composition is a mixture of the molding and the mixed metal oxide.
35. A process for preparing the composition according to any one of embodiments 1 to 34, the process comprising
   (i) providing a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM;
   (ii) providing a mixed metal oxide comprising chromium, zinc, and aluminum;
   (iii) mixing the molding provided according to (i) with the mixed metal oxide provided according to (ii), obtaining the composition.
36. The process of embodiment 35, wherein the molding comprising a zeolitic material having an AEI-type framework structure displays a total amount of acid sites as determined by NH₃-TPD in the range of from 0.05 to 1.8 mmol/g, preferably from 0.1 to 1.5 mmol/g, more preferably from 0.3 to 1.3 mmol/g, more preferably from 0.5 to 1.2 mmol/g, more preferably from 0.6 to 1.1 mmol/g, more preferably from 0.7 to 1 mmol/g, more preferably from 0.8 to 0.95 mmol/g, and more preferably from 1.35 to 1.45 mmol/g, wherein preferably the NH₃-TPD is obtained according to the method described in the experimental section of the present application.
37. The process of embodiment 35 or 36, wherein the molding comprising a zeolitic material having an AEI-type framework structure displays a NH₃-TPD desorption spectrum, preferably a deconvoluted NH₃-TPD desorption spectrum, comprising a peak in the range of from 300 to 550° C., preferably from 330 to 520° C., more preferably from 350 to 500° C., more preferably from 380 to 480° C., more preferably from 400 to 450° C., more preferably from 410 to 430° C., and more preferably from 415 to 420° C., wherein preferably the NH₃-TPD is obtained according to the method described in the experimental section of the present application.
38. The process of embodiment 37, wherein the integration of the peak affords an amount of acid sites in the range of from 0.01 to 0.3 mmol/g, preferably from 0.02 to 0.2 mmol/g, more preferably from 0.03 to 0.15 mmol/g, more preferably from 0.04 to 0.12 mmol/g, more preferably from 0.05 to 0.1 mmol/g, and more preferably from 0.06 to 0.08 mmol/g.
39. The process of any one of embodiments 35 to 38, wherein providing a molding according to (i) comprises
   (i.1) providing a zeolitic material having an AEI-type framework structure, wherein the zeolitic material has a framework structure comprising Si, a trivalent element X, and oxygen;
   (i.2) optionally impregnating the zeolitic material obtained from (i.1) with a source of the one or more alkaline earth metals AEM;
   (i.3) preparing a molding comprising the impregnated zeolitic material obtained from (i.2) or the zeolitic material from (i.1) and optionally a binder material.
40. The process of any one of embodiments 35 to 39, wherein X is selected from the group consisting of Al, B, In, Ga, and mixtures of two or more thereof, X preferably being Al and/or B, and more preferably being Al.
41. The process of any one of embodiments 35 to 40, wherein the zeolitic material having an AEI-type framework structure displays a total amount of acid sites as determined by NH₃-TPD in the range of from 0.1 to 3 mmol/g, preferably from 0.3 to 2.5 mmol/g, more preferably from 0.5 to 2.2 mmol/g, more preferably from 0.8 to 2 mmol/g, more preferably from 1 to 1.8 mmol/g, more preferably from 1.1 to 1.7 mmol/g, more preferably from 1.2 to 1.6 mmol/g, more preferably from 1.3 to 1.5 mmol/g, and more preferably from 1.35 to 1.45 mmol/g, wherein preferably the NH₃-TPD is obtained according to the method described in the experimental section of the present application.
42. The process of any one of embodiments 35 to 41, wherein the zeolitic material having an AEI-type framework structure displays a NH₃-TPD desorption spectrum, preferably a deconvoluted NH₃-TPD desorption spectrum, comprising a peak in the range of from 400 to 600° C., preferably from 430 to 600° C., more preferably from 450 to 580° C., more preferably from 480 to 550° C., more preferably from 500 to 530° C., and more preferably from 510 to 520° C., wherein preferably the NH₃-TPD is obtained according to the method described in the experimental section of the present application.
43. The process of embodiment 42, wherein the integration of the peak affords an amount of acid sites in the range of from 0.05 to 1.5 mmol/g, preferably from 0.1 to 1.2 mmol/g, more preferably from 0.2 to 1 mmol/g, more preferably from 0.3 to 0.9 mmol/g, more preferably from 0.4 to 0.8 mmol/g, more preferably from 0.5 to 0.7 mmol/g, and more preferably from 0.55 to 0.65 mmol/g.
44. The process of any one of embodiments 35 to 43, wherein the one or more alkaline earth metals AEM is one or more of Be, Mg, Ca, Sr and Ba, wherein the one or more alkaline earth metals AEM preferably comprises, more preferably is Mg.
45. The process of any one of embodiments 39 to 44, wherein in the framework structure of the zeolitic material provided according to (i.1), the molar ratio Si:X, calculated as SiO₂:X₂O₃, is in the range of from 4 to 300, preferably from 6 to 150, more preferably from 8 to 100, more preferably from 10 to 50, more preferably from 11 to 30, more preferably from 12 to 20, more preferably from 12.5 to 16, and more preferably from 13 to 14.
46. The process of any one of embodiments 39 to 45, wherein at least 95 weight-%, preferably at least 98 weight-%, more preferably at least 99 weight-%, more preferably at least 99.5 weight-%, more preferably at least 99.9 weight % of the framework structure of the zeolitic material provided according to (i.1) consist of Si, X, O, and H.
47. The process of any one of embodiments 39 to 46, wherein at most 1 weight-%, preferably at most 0.1 weight-%, more preferably at most 0.01 weight-%, more preferably from to 0.001 weight-% of the framework structure of the zeolitic material provided according to (i.1) consist of phosphorous.
48. The process of any one of embodiments 39 to 47, wherein at least 95 weight-%, preferably at least 98 weight-%, more preferably at least 99 weight-%, more preferably at least 99.5 weight-%, more preferably at least 99.9 weight-% of the zeolitic material provided according to (i.1) consist of Si, X, O, H, and the one or more alkali metals AM and/or the one or more alkaline earth metals AEM.
49. The process of any one of embodiments 35 to 48, wherein the one or more alkali metals AM is one or more of Li, Na, K, Rb, and Cs, wherein the one or more alkali metals AM preferably comprises, more preferably is Na.
50. The process of any one of embodiments 39 to 49, wherein the source of the one or more alkaline earth metals AEM according to (i.2) is a salt of the one or more alkaline earth metals.
51. The process of any one of embodiments 39 to 50, wherein the source of the one or more alkaline earth metals AEM according to (i.2) is a salt of the one or more alkaline earth metals dissolved in one or more solvents, preferably dissolved in water.
52. The process of any one of embodiment 39 to 51, wherein impregnating the zeolitic material according to (i.2) comprises one or more of wet-impregnating the zeolitic material and spray-impregnating the zeolitic material, preferably spray-impregnating the zeolitic material.
53. The process of any one of embodiments 39 to 52, wherein (i.2) further comprises calcining the zeolitic material obtained from impregnation, optionally after drying the zeolitic material obtained from impregnation, wherein the calcining is preferably carried out in a gas atmosphere having a temperature in the range of from 400 to 650° C., preferably in the range of from 450 to 600° C., wherein the gas atmosphere is preferably nitrogen, oxygen, air, lean air, or a mixture of two or more thereof, wherein, if drying is carried out prior to calcining, the drying is preferably carried out in a gas atmosphere having a temperature in the range of from 75 to 200° C., preferably in the range of from 90 to 150° C., wherein the gas atmosphere is preferably nitrogen, oxygen, air, lean air, or a mixture of two or more thereof.
54. The process of any one of embodiments 39 to 53, wherein at least 95 weight-%, preferably at least 98 weight-%, more preferably at least 99 weight-%, more preferably at least 99.5 weight-%, more preferably at least 99.9 weight-% of the impregnated zeolitic material obtained from (i.2) consist of Si, X, 0, H, and the one or more alkali metals AM and/or the one or more alkaline earth metals AEM.
55. The process of any one of embodiments 35 to 54, wherein the one or more alkali metals AM are comprised in the zeolitic material having an AEI-type framework structure in a total amount in the range of from 0.01 to 7 wt.-% calculated as the element and based on 100 wt.-% of the total amount of Si, calculated as SiO₂, contained in the zeolitic material, preferably from 0.05 to 5 wt.-%, more preferably from 0.1 to 4 wt.-%, more preferably from 0.5 to 3.8 wt.-%, more preferably from 1 to 3.6 wt.-%, more preferably from 1.5 to 3.4 wt.-%, more preferably from 2 to 3.2 wt.-%, more preferably from 2.3 to 3 wt.-%, more preferably from 2.5 to 2.9 wt.-%, and more preferably from 2.6 to 2.8 wt.-%.
56. The process of any one of embodiments 35 to 55, wherein the zeolitic material comprises the one or more alkaline earth metals AEM, calculated as the element, in a total amount in the range of from 0.1 to 5 weight-%, preferably in the range of from 0.4 to 3 weight-%, more preferably in the range of from 0.75 to 2 weight-%, based on the total weight of the zeolitic material.
57. The process of any one of embodiments 39 to 56, wherein preparing a molding according to (i.3) comprises
   (i.3.1) preparing a mixture of the impregnated zeolitic material obtained from (i.2) and a source of a binder material;
   (i.3.2) subjecting the mixture prepared according to (i.3.1) to shaping.
58. The process of embodiment 57, wherein the source of a binder material is one or more of a source of graphite, a source of silica, a source of titania, a source of zirconia, a source of alumina and a source of a mixed oxide of two or more of silicon, titanium, zirconium and aluminum, wherein the source of a binder material preferably comprises, more preferably is a source of silica, wherein the source of silica preferably comprises one or more of a colloidal silica, a fumed silica, and a tetraalkoxysilane, more preferably comprises a colloidal silica.
59. The process of embodiment 57 or 58, wherein the mixture prepared according to (i.3.1) further comprises a pasting agent, wherein the pasting agent preferably comprises one or more of an organic polymer, an alcohol and water, wherein the organic polymer is preferably one or more of a carbohydrate, a polyacrylate, a polymethacrylate, a polyvinyl alcohol, a polyvinylpyrrolidone, a polyisobutene, a polytetrahydrofuran, and a polyethlyene oxide, wherein the carbohydrate is preferably one or more of cellulose and cellulose derivative, wherein the cellulose derivative is preferably a cellulose ether, more preferably a hydroxyethyl methylcellulose, wherein more preferably, the pasting agent comprises one or more of water and a carbohydrate.
60. The process of any one of embodiments 57 to 59, wherein subjecting to shaping according to (i.3.2) comprises subjecting the mixture prepared according to (i.3.1) to spray-drying, to spray-granulation, or to extrusion, preferably to extrusion.
61. The process of any one of embodiments 57 to 60, further comprising
   (i.3.3) calcining the molding obtained from (i.3.2), optionally after drying, wherein the calcining is preferably carried out in a gas atmosphere having a temperature in the range of from 500 to 950° C., preferably in the range of from 600 to 900° C., more preferably in the range of from 650 to 850° C., more preferably in the range of from 700 to 820° C., and more preferably in the range of from 750 to 800° C.,
   wherein the gas atmosphere is preferably nitrogen, oxygen, air, lean air, or a mixture of two or more thereof, wherein, if drying is carried out prior to calcining, the drying is preferably carried out in a gas atmosphere having a temperature in the range of from 75 to 200° C., preferably in the range of from 90 to 150° C., wherein the gas atmosphere is preferably nitrogen, oxygen, air, lean air, or a mixture of two or more thereof.
62. The process of any one of embodiment 35 to 61, wherein providing the mixed metal oxide according to (ii) comprises
   (ii.1) co-precipitating a precursor of the mixed metal oxide from sources of the chromium, the zinc, and the aluminum;
   (ii.2) washing the precursor obtained from (ii.1);
   (ii.3) drying the washed precursor obtained from (ii.2);
   (ii.4) calcining the washed precursor obtained from (ii.3).
63. The process of embodiment 62, wherein co-precipitating a precursor according to (ii.1) comprises
   (ii.1.1) preparing a mixture comprising water and the sources of the zinc and the aluminum, wherein the sources of the zinc and the aluminum preferably comprises one or more of a zinc salt and an aluminum salt, wherein more preferably, the zinc salt is a zinc nitrate, preferably a Zn(II) nitrate, and the aluminum salt is an aluminum nitrate, preferably an aluminum(III) nitrate;
   (ii.1.2) adding a precipitation agent to the mixture prepared according to (ii.1.1), wherein the precipitation agent preferably comprises an ammonium carbonate, more preferably an ammonium carbonate dissolved in water;
   (ii.1.3) subjecting the mixture obtained from (ii.1.2) to heating to a temperature of the mixture in the range of from 50 to 90° C., preferably in the range of from 60 to 80° C., and keeping the mixture at this temperature for a period of time, wherein the period of time is preferably in the range of from 0.1 to 12 h, more preferably in the range of from 0.5 to 6 h;
   (ii.1.4) optionally drying the mixture obtained from (ii.1.3), preferably in a gas atmosphere having a temperature in the range of from 75 to 200° C., preferably in the range of from 90 to 150° C., wherein the gas atmosphere is preferably oxygen, air, lean air, or a mixture of two or more thereof;
   (ii.1.5) calcining the mixture obtained from (ii.1.3) or from (ii.1.4), preferably from (ii.1.4), preferably in a gas atmosphere having a temperature in the range of from 300 to 900° C., preferably in the range of from 350 to 800° C., wherein the gas atmosphere is preferably oxygen, air, lean air, or a mixture of two or more thereof, obtaining the mixed metal oxide;
   (ii.1.6) preparing a mixture comprising water and the source of the chromium, wherein the source of the chromium preferably comprises a chromium salt, wherein more preferably, the chromium salt is a chromium nitrate, preferably a chromium(III) nitrate;
   (ii.1.7) impregnating the calcined mixed metal oxide obtained from (ii.1.5) with the mixture obtained from (ii.1.6), preferably by incipient wetness;
   (ii.1.8) optionally drying the mixture obtained from (ii.1.7), preferably in a gas atmosphere having a temperature in the range of from 75 to 200° C., preferably in the range of from 90 to 150° C., wherein the gas atmosphere is preferably oxygen, air, lean air, or a mixture of two or more thereof;
   (ii.1.9) calcining the mixture obtained from (ii.1.7) or from (ii.1.8), preferably from (ii.1.8), preferably in a gas atmosphere having a temperature in the range of from 300 to 900° C., preferably in the range of from 350 to 800° C., wherein the gas atmosphere is preferably oxygen, air, lean air, or a mixture of two or more thereof, obtaining the impregnated mixed metal oxide.
64. The process of embodiment 63, wherein according to (ii.1.5) and/or (ii.1.9), preferably according to (ii.1.5) and (ii.1.9), the mixture is calcined at a temperature in the range of from 350 to 440° C., preferably in the range of from 375 to 425° C.
65. The process of embodiment 63, wherein according to (ii.1.5) and/or (ii.1.9), preferably according to (ii.1.5) and (ii.1.9), the mixture is calcined at a temperature in the range of from 450 to 550° C., preferably in the range of from 475 to 525° C.
66. The process of embodiment 63, wherein according to (ii.1.5) and/or (ii.1.9), preferably according to (ii.1.5) and (ii.1.9), the mixture is calcined at a temperature in the range of from 700 to 800° C., preferably in the range of from 725 to 775° C.
67. The process of any one of embodiments 63 to 66, wherein in the mixture prepared in (ii.1.1), the weight ratio of the zinc, calculated as element, relative to the aluminum, calculated as element, is in the range of from 0.5 to 2, preferably in the range of from 0.8 to 1.7, more preferably in the range of from 0.9 to 1.5, more preferably in the range of from 1 to 1.25, and more preferably in the range of from 1.1 to 1.15.
68. The process of any one of embodiments 63 to 67, wherein in the mixture prepared in (ii.1.7), the weight ratio of the aluminum, calculated as element, relative to the chromium, calculated as element, is in the range of from 1.5 to 4, preferably in the range of from 2 to 3.5, more preferably in the range of from 2.3 to 3.2, and more preferably in the range of from 2.5 to 3.
69. A molding, obtainable or obtained by a process according to any one of embodiments 39 to 61.
70. A mixed metal oxide, obtainable or obtained by a process according to any one of embodiments 62 to 68.
71. A composition, obtainable or obtained by a process according to any one of embodiments 35 to 68.
72. Use of a composition according to any one of embodiments 1 to 34 or 71 as a catalyst or as a catalyst component, preferably for preparing C2 to C4 olefins, more preferably for preparing C2 to C4 olefins from a synthesis gas comprising hydrogen and carbon monoxide, wherein the C2 to C4 olefins is preferably one or more of ethene and propene, more preferably propene, wherein preparing the C2 to C4 olefins is preferably carried out as a one-step process.
73. A process for preparing C2 to C4 olefins from a synthesis gas comprising hydrogen and carbon monoxide, the process comprising
   (1) providing a gas stream which comprises a synthesis gas stream comprising hydrogen and carbon monoxide;
   (2) providing a catalyst comprising a composition according to any one of embodiments 1 to 34 or 71;
   (3) bringing the gas stream provided in (1) in contact with the catalyst provided in (2), obtaining a reaction mixture stream comprising C2 to C4 olefins.
74. The process of embodiment 73, wherein in the synthesis gas stream provided in (1), the molar ratio of hydrogen relative to carbon monoxide is in the range of from 0.1 to 10, preferably in the range of from 0.2 to 5, more preferably in the range of from 0.25 to 2.
75. The process of embodiment 73 or 74, wherein at least 99 volume-%, preferably at least 99.5 volume-%, more preferably at least 99.9 volume-% of the synthesis gas stream according to (1) consist of hydrogen and carbon monoxide.
76. The process of any one of embodiments 73 to 75, wherein at least 80 volume-%, preferably at least 85 volume-%, more preferably at least 90 volume-%, more preferably from 90 to 99 volume-% of the gas stream provided in (1) consist of the synthesis gas stream.
77. The process of any one of embodiments 73 to 76, wherein the gas stream provided in (1) further comprises one or more inert gas preferably comprising, more preferably being one or more of nitrogen and argon.
78. The process of embodiment 77, wherein in the gas stream provided in (1), the volume ratio of the one or more inter gases relative to the synthesis gas stream is in the range of from 1:20 to 1:2, preferably in the range of from 1:15 to 1:5, more preferably in the range of from 1:12 to 1:8.
79. The process of embodiment 77 or 78, wherein at least 99 volume-%, preferably at least 99.5 volume-%, more preferably at least 99.9 volume-% of the gas stream provided in (1) consist of the synthesis gas stream and the one or more inert gases.
80. The process of any one of embodiments 73 to 79, wherein according to (3), the gas stream is brought in contact with the catalyst at a temperature of the gas stream in the range of from 200 to 550° C., preferably in the range of from 250 to 525° C., more preferably in the range of from 300 to 500° C.
81. The process of any one of embodiments 73 to 80, wherein according to (3), the gas stream is brought in contact with the catalyst at a pressure of the gas stream in the range of from 10 to 40 bar(abs), preferably in the range of from 12.5 to 30 bar(abs), more preferably in the range of from 15 to 25 bar(abs).
82. The process of any one of embodiments 73 to 81, wherein the catalyst provided in (2) is comprised in a reactor tube, and wherein bringing the gas stream provided in (1) in contact with the catalyst provided in (2) according to (3) comprises passing the gas stream as feed stream into the reactor tube and through the catalyst bed comprised in the reactor tube, obtaining the reaction mixture stream comprising C2 to C4 olefins, said process further comprising removing the reaction mixture stream from the reactor tube.
83. The process of embodiment 82, wherein according to (3), the gas stream is brought in contact with the catalyst at a gas hourly space velocity in the range of from 100 to 25,000 h⁻¹, preferably in the range of from 500 to 20,000 h⁻¹, more preferably in the range of from 1,000 to 10,000 h⁻¹, wherein the gas hourly space velocity is defined as the volume flow rate of the gas stream brought in contact with the catalyst divided by the volume of the catalyst bed.
84. The process of any one of embodiments 73 to 83, wherein prior to (3), the catalyst provided in (2) is activated.
85. The process of embodiment 84, wherein activating the catalyst comprises bringing the catalyst in contact with a gas stream comprising hydrogen and an inert gas, wherein preferably from 1 to 50 volume-%, more preferably from 2 to 35 volume-%, more preferably from 5 to 20 volume-% of the gas stream consist of hydrogen, and wherein the inert gas preferably comprises one or more of nitrogen and argon, more preferably nitrogen.
86. The process of embodiment 85, wherein at least 98 volume-%, preferably at least 99 volume-%, more preferably at least 99.5 volume-% of the gas stream comprising hydrogen consist of hydrogen and the inert gas.
87. The process of embodiment 85 or 86, wherein the gas stream comprising hydrogen is brought in contact with the catalyst at a temperature of the gas stream in the range of from 200 to 400° C., preferably in the range of from 250 to 350° C., more preferably in the range of from 275 to 325° C.
88. The process of any one of embodiments 85 or 87, wherein the gas stream comprising hydrogen is brought in contact with the catalyst at a pressure of the gas stream in the range of from 1 to 50 bar(abs), preferably in the range of from 5 to 40 bar(abs), more preferably in the range of from 10 to 30 bar(abs).
89. The process of any one of embodiments 85 to 88, wherein the catalyst provided in (2) is comprised in a reactor tube, and wherein prior to (3), bringing the gas stream comprising hydrogen in contact with the catalyst provided in (2) comprises passing the gas stream comprising hydrogen into the reactor tube and through the catalyst bed comprised in the reactor tube.
90. The process of embodiment 89, wherein the gas stream comprising hydrogen is brought in contact with the catalyst at a gas hourly space velocity in the range of from 500 to 15,000 h⁻¹, preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed.
91. The process of any one of embodiments 84 to 90, wherein activating the catalyst further comprises bringing the catalyst in contact with a synthesis gas stream comprising hydrogen and carbon monoxide, wherein in the synthesis gas stream the molar ratio of hydrogen relative to carbon monoxide is preferably in the range of from 0.1 to 10, more preferably in the range of from 0.2 to 5, more preferably in the range of from 0.25 to 2, wherein preferably at least 99 volume-%, more preferably at least 99.5 volume-%, more preferably at least 99.9 volume-% of the synthesis gas stream according to (1) consist of hydrogen and carbon monoxide.
92. The process of embodiment 91, wherein the synthesis gas stream comprising hydrogen and carbon monoxide used for activating the catalyst is the synthesis gas stream provided in (1).
93. The process of embodiment 91 or 92, wherein for activating the catalyst, the synthesis gas stream comprising hydrogen and carbon monoxide is brought in contact with the catalyst at a temperature of the gas stream in the range of from 100 to 300° C., preferably in the range of from 150 to 275° C., more preferably in the range of from 200 to 250° C.
94. The process of any one of embodiments 91 or 93, wherein for activating the catalyst, the synthesis gas stream comprising hydrogen and carbon monoxide is brought in contact with the catalyst at a pressure of the gas stream in the range of from 10 to 50 bar(abs), preferably in the range of from 15 to 35 bar(abs), more preferably in the range of from 20 to 30 bar(abs).
95. The process of any one of embodiments 91 to 94, wherein the catalyst provided in (2) is comprised in a reactor tube, and wherein for activating the catalyst, bringing the synthesis gas stream comprising hydrogen and carbon monoxide in contact with the catalyst provided in (2) comprises passing the synthesis gas stream comprising hydrogen and carbon monoxide into the reactor tube and through the catalyst bed comprised in the reactor tube.
96. The process of embodiment 95, wherein the synthesis gas stream comprising hydrogen and carbon monoxide is brought in contact with the catalyst at a gas hourly space velocity in the range of from 500 to 15,000 h⁻¹, preferably in the range of from 1,000 to 10,000 h⁻¹, more preferably in the range of from 2,000 to 8,000 h⁻¹, wherein the gas hourly space velocity is defined as the volume flow rate of the gas stream brought in contact with the catalyst divided by the volume of the catalyst bed.
97. The process of any one of embodiments 91 to 96, wherein for activating the catalyst prior to (3), bringing the synthesis gas stream comprising hydrogen and carbon monoxide in contact with the catalyst provided in (2) is carried out prior to bringing the catalyst in contact with a gas stream comprising hydrogen and an inert gas according to any one of embodiments 73 to 78.
98. The process of any one of embodiments 73 to 97, wherein the C2 to C4 olefins comprise, preferably consist of ethene, propene, and a butene, wherein the butene is preferably 1-butene.
99. The process of embodiment 98 wherein in the reaction mixture obtained according to (3), the molar ratio of propene relative to ethene is greater than 1 and the molar ratio of ethene relative to the butene is greater than 1.
100. The process of any one of embodiments 73 to 99, wherein the conversion of the synthesis gas to the C2 to C4 olefins exhibits a selectivity towards the C2 to C4 olefins of at least 30%.

### DESCRIPTION OF THE FIGURES

FIG. 1 displays the results from catalytic testing of the mixed metal oxide catalysts from Reference Example 1 “CrZn” and Comparative Example 1 “ZrZn” in the conversion of synthesis gas to methanol and dimethylether.

FIG. 2 displays the results from extended catalytic testing of the mixed metal oxide catalyst from Reference Example 1 in the conversion of synthesis gas to methanol and dimethylether.

FIG. 3 displays the results from catalytic testing of the zeolite catalysts from Reference Example 2 and Comparative Example 2 in the conversion of synthesis gas and methanol to C2 to C4 olefins.

FIG. 4 displays the results from extended catalytic testing of the zeolite catalyst from Reference Example 2 in the conversion of synthesis gas and methanol to olefins.

### EXAMPLES

### Determination of the BET Specific Surface Area

The BET specific surface area was determined via nitrogen physisorption at 77 K according to the method disclosed in DIN 66131.

### Determination of Selectivities and Yields

The selectivity of a given product compound, in %, referred to in the following as “S_{N_}SubstanceA”, is a normalized selectivity S_{N} and is calculated as follows:

S_{N_}SubstanceA/%=S_SubstanceA/%*Fact_normS

wherein
S_SubstanceA/%=selectivity of substance A
Fact_normS=normalization factor, used to achieve a sum of the selectivities of 100%

### a) S_SubstanceA

The selectivity of substance A, S_SubstanceA, is defined as

S_SubstanceA/%=(Y_SubstanceA/X_CO(IntStd))*100

wherein
Y_SubstanceA=yield of substance A
X_CO(IntStd)=conversion of CO calculated based on an internal standard, in the present case an inert liner (Argon)

### a.1) Y_SubstanceA

The yield of substance A, Y_SubstanceA, is defined

Y_SubstanceA/%=(R(C)_SubstanceA/R(C)_CO_in)*100

wherein
R(C)_SubstanceA=the rate of carbon of substance A, determined in g/h via gas chromatography
R(C)_CO_in =the rate of carbon monoxide CO which is fed to the reactor, determined in (g carbon)/h

### a.2) X_CO(IntStd)

The conversion of CO, X_CO(IntStd), is defined as

X_CO(IntStd)=(1−(RA_CO/Arout)/(RA_CO/AroutRef))*100

wherein
RA_CO/Arout=rate of CO determined via gas chromatography, divided by the rate of the inert liner Ar determined via GC
RA_CO/AroutRef=rate of CO/reference determined via gas chromatography, divided by the rate of inert liner Ar/reference determined via gas chromatography (i.e. rate of CO at the inlet divided by rate of Ar at the inlet

### b) Fact_normS

The normalization factor, Fact_normS, is defined as

Fact_normS=100/((Sum of all S)−(S_starting material))

wherein
Sum of all S=sum of all selectivities measured at the outlet of the reactor (which would include the selectivites of starting material at the out let of the conversion is not 100%)
S_starting material=selectivites of the starting materials (if conversion is 100%, the value would be 0%)

### Temperature Programmed Desorption of Ammonia (NH₃-TPD)

The temperature-programmed desorption of ammonia (NH₃-TPD) was conducted in an automated chemisorption analysis unit (Micromeritics AutoChem II 2920) having a thermal conductivity detector. Continuous analysis of the desorbed species was accomplished using an online mass spectrometer (OmniStar QMG200 from Pfeiffer Vacuum). The sample (0.1 g) was introduced into a quartz tube and analysed using the program described below. The temperature was measured by means of a Ni/Cr/Ni thermocouple immediately above the sample in the quartz tube. For the analyses, He of purity 5.0 was used. Before any measurement, a blank sample was analysed for calibration.
1. Preparation: Commencement of recording; one measurement per second. Wait for 10 minutes at 25° C. and a He flow rate of 30 cm³/min (room temperature (about 25° C.) and 1 atm); heat up to 600° C. at a heating rate of 20 K/min; hold for 10 minutes. Cool down under a He flow (30 cm³/min) to 100° C. at a cooling rate of 20 K/min (furnace ramp temperature); Cool down under a He flow (30 cm³/min) to 100° C. at a cooling rate of 3 K/min (sample ramp temperature).
2. Saturation with NH₃: Commencement of recording; one measurement per second. Change the gas flow to a mixture of 10% NH₃ in He (75 cm³/min; 100° C. and 1 atm) at 100° C.; hold for 30 minutes.
3. Removal of the excess: Commencement of recording; one measurement per second.

Change the gas flow to a He flow of 75 cm³/min (100° C. and 1 atm) at 100° C.; hold for 60 min.
4. NH₃-TPD: Commencement of recording; one measurement per second. Heat up under a He flow (flow rate: 30 cm³/min) to 600° C. at a heating rate of 10 K/min; hold for 30 minutes.
5. End of measurement.

Desorbed ammonia was measured by means of the online mass spectrometer, which demonstrates that the signal from the thermal conductivity detector was caused by desorbed ammonia. This involved utilizing the m/z=16 signal from ammonia in order to monitor the desorption of the ammonia. The amount of ammonia adsorbed (mmol/g of sample) was ascertained by means of the Micromeritics software through integration of the TPD signal with a horizontal baseline.

### Catalyst Testing Setup

The catalytic conversions in the examples were investigated in a fixed catalyst bed consisting of catalyst split fraction (oxide or zeolite). The reactions were performed in the gas phase using a 16-fold unit with stainless steel reactors. Catalysts were tested with a particle size fraction of 250-315 μm and catalyst volumes of 1 mL, 0.9 mL, 0.6 mL, and 0.4 mL.

The reactions temperatures were varied from 350-425° C., the pressure varied between 25, 30, and 35 bar. Composition of feedstock included a mixtures of H₂/CO/MeOH/DME for zeolites, and H₂/CO for oxide catalysts.

### Reference Example 1: Preparation of a Mixed Metal Oxide of Cr, Zn, and Al

108 g of Al(NO₃)₃*9 H₂O (Honeywell, 98%) and 40 g Zn(NO₃)₂*6 H₂O (Honeywell, 98%) were dissolved in 1 L of distilled water under stirring. The solution displaying a pH of 1.68 was then placed in a vessel and heated under stirring to 70° C. 388 g of a 20 wt % aqueous solution of (NH₄)₂CO₃ (Aldrich) were then added dropwise to the mixture over 1 h until a pH of 7 was reached. The mixture was then further stirred at 70° C. for 2.5 h, during which a white solid precipitated from the solution. The solid was then filtered off and washed with 9 liters of distilled water until the washwater displayed a conductivity of <10 pS. The filter cake was then dried over night at 110° C., and then heated in a muffle oven in 4 h to 500° C. and calcined at that temperature for 1 h for obtaining a Zn/AI mixed metal oxide.

21.76 g of Cr(NO₃)₃*9 H₂O [[2.83 g Cr]] (Sigma Aldrich, 99%) were dissolved in 30.6 ml of distilled water. 26.5 g of the calcined Zn/AI mixed metal oxide were then mixed with the aqueous chrome nitrate solution for impregnation thereof, and the resulting slurry was then dried over night at 110° C., wherein the slurry was repeatedly mixed during the drying step for ensuring the impregnation of the mixed metal oxide with the chrome nitrate solution. The impregnated mixed metal oxide was then heated in a muffle oven in 4 h to 500° C. and calcined at that temperature for 1 h. The calcined powder was then sieved through a 1 mm sieve and the powder then pressed with 35 bar pressure in a Shell-Test press to platelets with a diameter of 2 cm. The platelets were then processed to a fraction of 315-500 μm.

Elemental analysis of the resulting Zn/Al/Cr mixed metal oxide afforded values of 24.7 wt.-% of Zn, 24.0 wt.-% of Al, and 11.3 wt.-% of Cr.

The BET surface area of the resulting Zn/Al/Cr mixed metal oxide was 113.45 m²/g.

### Comparative Example 1: Preparation of a Mixed Metal Oxide of Zr and Zn

130 g of zirconium(IV)oxynitrate hydrate (Sigma Aldrich 99%) and 48.5 g Zn(NO₃)₂*6 H₂O (Honeywell, 98%) were dissolved in 0.8 L of distilled water under stirring. The solution displaying a pH of 0.03 was then placed in a vessel and heated under stirring to 70° C. 422 g of a 20 wt % aqueous solution of Na₂CO₃ (Bernd Kraft) were then added dropwise to the mixture, wherein after 80 min of precipitation (pH=1.6) to solution turned into a gel, after which the precipitation was interrupted and the gel was further mixed with the aid of a spatula, and 100 ml of distilled water were stirred in, followed by further precipitating while stirring the mixture at a high stirring rate (450 rm) until a pH of 7 was eventually reached after 2.5 h of precipitation. The mixture was then further stirred at 100 rpm over night at room temperature. The solid was then filtered off and washed with 52 liters of distilled water until the washwater displayed a conductivity of <10 μS. The filter cake was then dried for 12 h at 110° C., and then heated in a muffle oven in 4 h to 500° C. and calcined at that temperature for 5 h for obtaining a Zr/Zn mixed metal oxide.

The Zr/Zn mixed metal oxide powder was then sieved through a 1 mm sieve and the powder then pressed with 35 bar pressure in a Shell-Test press to platelets with a diameter of 2 cm. The platelets were then processed to a fraction of 315-500 μm.

Elemental analysis of the resulting Zr/Zn mixed metal oxide afforded values of 53 wt.-% of Zr and 16.4 wt.-% of Zn.

The BET surface area of the resulting Zr/Zn mixed metal oxide was 29.97 m²/g.

### Reference Example 2: Preparation of Extrudate of an AEI Zeolitic Material Calcined at 800° C.

a) Providing an AEI zeolitic material.
20.194 kg of distilled water were placed in a 60 L autoclave reactor and stirred at 200 rpm. 2.405 kg of a solution of 50 wt.-% NaOH in distilled water were then added followed by the addition of 6.670 kg of 1,1,3,5-tetramethylpiperidinium hydroxide. 560 g of zeolite Y seeds (NH₄-zeolite Y; CBV-500 from Zeolyst) were then suspended in 3 L of distilled water and the suspension was the added to the reactor while stirring, after which 7.473 kg of Ludox® AS40 (Grace; colloidal silica; aqueous solution, 40 weight-%) were added. The resulting mixture displaying molar ratios of 1.00 SiO₂: 0.30 Na₂O: 0.17 template: 0.19 zeolite Y: 41.5H₂O was further stirred for 30 min at room temperature, after which the reactor was closed and the reaction mixture was heated under autogenous pressure in 1.5 h to 160° C. and subsequently maintained at that temperature for 48 h while further stirring.
The resulting suspension was filled into five 10 L canisters and the suspension allowed to settle, after which the clear supernatant was decanted off. The solid residue was placed in a filter and washed with distilled water to <200 μS. The filter cake was then dried at 120° C. over night to afford 1.1848 kg of a crystalline solid, which was subsequently heated at 2° C./min to 500° C. and calcined at that temperature for 5 hours under air. After said calcination, the calcined zeolitic material was subject to a further calcination step, wherein it was heated at 2° C./min to 550° C. and calcined at that temperature for 5 h to afford 1.0810 kg of the sodium form of a zeolitic material. X-ray diffraction analysis of the zeolitic material revealed an AEI type framework structure. The Na-AEI zeolite displayed a BET surface area as obtained from the nitrogen isotherms of 506 m²/g and a Langmuir surface area of 685 m²/g.
Elemental analysis of the resulting Na-AEI zeolite afforded values of 34 wt.-% of Si, 5.1 wt.-% of Al, and 2 wt.-% of Na. Accordingly the zeolite displayed an SiO₂: Al₂O₃ molar ratio of 12.9.
NH₃-TPD analysis of the Na-AEI zeolite afforded a total amount of acid sites of 1.4 mmol/g, wherein the deconvoluted desorption spectrum included a peak at 515° C. having an amount of acid sites of 0.6 mmol/g.

### b) Preparing an Extrudate Comprising the AEI Zeolitic Material

Materials Used:

| | | |
|---|---|---|
| Na-AEI zeolitic material, according to a) above: | 80.0 | g |
| Ludox ® AS40 (Grace; colloidal silica; aqueous | 50.0 | g |
| solution, 40 weight-%): | | |
| Walocel | 5.0 | g |
| Deionized water | 92.0 | g |

The zeolitic material, the Ludox®, and the Walocel were kneaded for 1 h, wherein the distilled water was added to the mixture in portions during kneading. The material obtained was extruded and strands of 1 mm diameter were formed. The strands obtained were dried over night at 120° C. and then calcined for 5 hours in air at 800° C. 92 g of product were obtained.
NH₃-TPD analysis of the extrudate afforded a total amount of acid sites of 0.871 mmol/g, wherein the deconvoluted desorption spectrum included a peak at 418° C. having an amount of acid sites of 0.07 mmol/g.

### Comparative Example 2: Preparation of Extrudate of a CHA Zeolitic Material Containing 1% Mg

### a) Providing an Na-CHA Zeolitic Material.

A zeolitic material having framework type CHA was prepared as follows:
2,040 kg of water were placed in a stirring vessel and 3,924 kg of a solution of 1-adamantyltrimethyl ammoniumhydroxide (20 weight-% aqueous solution) were added thereto under stirring. 415.6 kg of a solution of sodium hydroxide (20 weight-% aqueous solution) were then added, followed by 679 kg of aluminum triisopropylate (Dorox® D 10, Ineos), after which the resulting mixture was stirred for 5 min. 7800.5 kg of a solution of colloidal silica (40 weight-% aqueous solution; Ludox® AS 40, Sigma Aldrich) were then added and the resulting mixture stirred for 15 min before being transferred to an autoclave. 1,000 kg of distilled water used for washing out the stirring vessel were added to the mixture in the autoclave, and the final mixture was then heated under stirring for 19 h at 170° C. The solid product was then filtered off and the filter cake washed with distilled water. The resulting filter cake was then dispersed in distilled water in a spray dryer mix tank to obtain a slurry with a solids concentration of approximately 24 weight-% and then spray dried, wherein the inlet temperature was set to 477-482° C. and the outlet temperature was measured to be 127-129° C., thus affording a spray dried powder of a zeolite having the CHA framework structure. The resulting material had a particle size distribution affording a Dv10 value of 1.4 micrometer, a Dv50 value of 5.0 micrometer, and a Dv90 value of 16.2 micrometer. The material displayed a BET specific surface area of 558 m²/g, a silica to alumina ratio of 34, a crystallinity of 105% as determined by powder X-ray diffraction. The sodium content of the product was determined to be 0.75 weight-% calculated as Na₂O.

### b) Providing a Mg-CHA Zeolitic Material

| | | |
|---|---|---|
| Na-CHA | 80 | g |
| Mg(NO₃)₂ × H₂O | 8.8 | g |
| Deionized water | 120 | g |

Mg(NO₃)₂×H₂O was dissolved in water and homogenized. The solution was added dropwise to the zeolitic material contained in a beaker, and the solution homogeneously distributed with the aid of a spatula. The impregnated zeolite was transferred in a porcelain bowl. The material was dried over night at 120° C. and then calcined for 5 hours at 500° C. 82 g of product were obtained. Elemental analysis of the zeolitic material showed an Mg content of 0.96 weight-%.

### c) Preparing an Extrudate Comprising the 1 Weight-% Mg-CHA Zeolitic Material

Materials Used:

| | | |
|---|---|---|
| 1% Mg-CHA zeolitic material, according to b) above: | 80.0 | g |
| Ludox ® AS40 (Grace; colloidal silica; aqueous | 50.0 | g |
| solution, 40 weight-%): | | |
| Walocel | 5.0 | g |
| Deionized water | 50 | g |

The zeolitic material, the Ludox®, the Walocel, and the water were kneaded for 1 h. The material obtained was extruded and strands of 1 mm diameter were formed. The strands obtained were dried over night at 120° C. and then calcined for 5 hours at 500° C. 94 g of product were obtained.

### Example 1: Catalytic Process for Preparing Methanol and Dimethylether from a Gas Stream Comprising Synthesis Gas

The conversion of synthesis gas over the mixed metal oxides of Reference Example 1 and Comparative Example 1 was tested in the catalyst testing setup described above. To this effect, an inlet gas stream containing 50 vol.-% of CO, 25 vol.-% of H₂, 9 vol.-% Ar, and N₂ gas make up was employed, wherein the reaction over the mixed metal oxide was conducted at a temperature of 350° C. and a pressure of 30 bara.

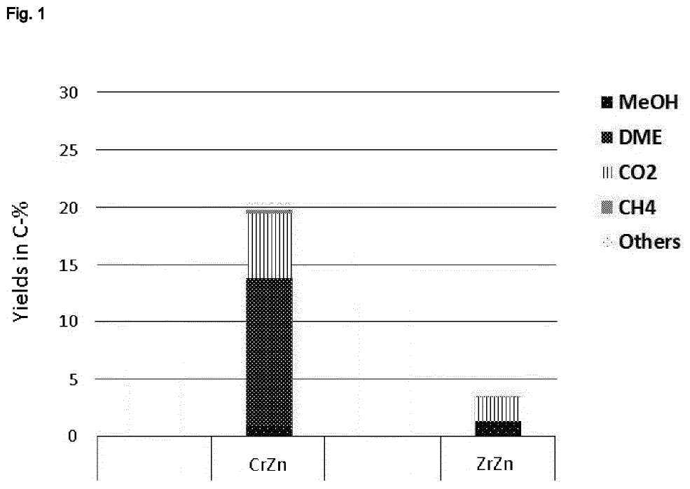

As may be taken from the results displayed in FIG. 1, the Zn/Al/Cr mixed metal oxide from Reference Example 1 provides a high conversion efficiency and a high selectivity towards dimethylether and methanol compared to the Zr/Zn mixed metal oxide from Comparative Example 1, which affords only a fraction of the yield of dimethylehter and methanol.

An extended test was performed with the Zn/Al/Cr mixed metal oxide from Reference Example 1, wherein the inlet gas stream contained 50 vol.-% of CO, 15 vol.-% of H₂, 9 vol.-% Ar, and N₂ gas make up, and the reaction was conducted at a temperature of 375° C. and at a pressure of 30 bara.

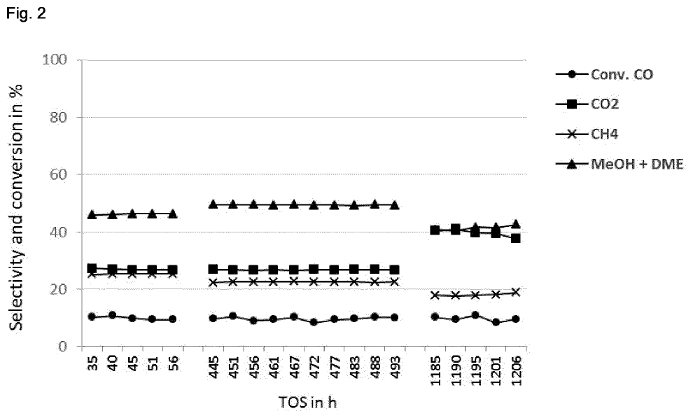

As may be taken from FIG. 2 displaying extended testing performed using the Zn/Al/Cr mixed metal oxide from Reference Example 1, the high selectivity towards methanol and dimethylether remains constant and even increases to a certain extent after long times on stream.

### Example 2: Process for Preparing C2 to C4 Olefins from a Gas Stream Comprising Synthesis Gas and Methanol

The conversion of synthesis gas and methanol over the zeolitic materials of Reference Example 2 and Comparative Example 2 was tested in the catalyst testing setup described above. To this effect, an inlet gas stream containing 44.95 vol.-% of CO, 44.95 vol.-% of H₂, 1 vol.-% of methanol, 9 vol.-% Ar, and N₂ gas make up was employed, wherein the reaction over the zeolitic material was conducted at a temperature of 400° C. and a pressure of 30 bara. The testing was performed at gas hourly space velocities of 1,500 and 2,500 h⁻¹.

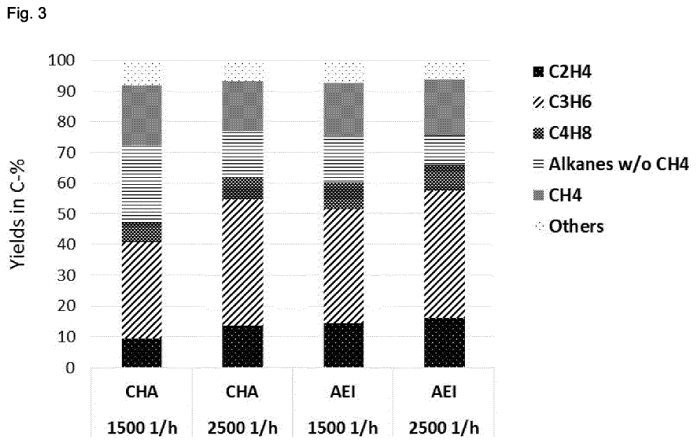

As may be taken from the results displayed in FIG. 3, although the zeolitic materials from Reference Example 2 and Comparative Example 2 both afforded high yields in ethylene, propene, and butene, the AEI zeolitic material from Reference Example 2 surprisingly showed higher yields than the Mg-CHA zeolitic material, in particular at the lower gas hourly space velocity of 1,500 h⁻¹. Said results are particularly unexpected given the fact that the AEI zeolitic material of Reference Example 2 does not contain any magnesium, which is known to improve the selectivity in C2 to C4 olefins.

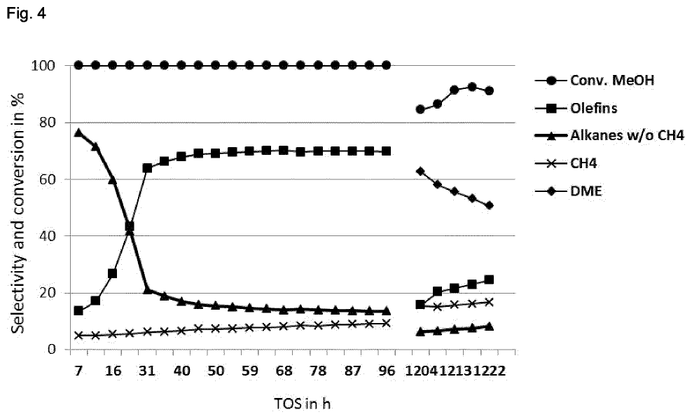

An extended test was performed with the AEI zeolitic material from Reference Example 2 under the same conditions as above. As may be taken from the results displayed in FIG. 4, the high yields in olefins are first achieved after a start up phase in which the reaction initially affords mainly alkanes. After said start up phase, the conversion rate of practically 100% and the high selectivity towards olefins remains constant for an extended period of time.

### CITED PRIOR ART

U.S. Pat. No. 4,049,573
Goryainova et al., in: Petroleum Chemistry, vol. 51, no. 3 (2011) pp. 169-173
Wan, V. Y., Methanol to Olefins/Propylene Technologies in China, Process Economics Programm, 261A (2013)
Li, J., X. Pan and X. Bao, Direct conversion of syngas into hydrocarbons over a core-shell Cr—Zn@SiO2@SAPO-34 catalyst, Chinese Journal of Catalysis vol. 36 no. 7 (2015), pp. 1131-1135
Unpublished patent application EP 17185280.9

1.-15. (canceled)

16. A composition comprising
a) a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM; and
b) a mixed metal oxide comprising chromium, zinc, and aluminum.

17. The composition of claim 16, wherein X is selected from the group consisting of Al, B, In, Ga, and mixtures of two or more thereof.

18. The composition of claim 16, wherein the one or more alkali metals AM is one or more of Li, Na, K, Rb, and Cs.

19. The composition of claim 16, wherein the one or more alkaline earth metals AEM is one or more of Be, Mg, Ca, Sr and Ba.

20. The composition of claim 16, wherein the one or more alkaline earth metals AEM is present in the zeolitic material at least partly in an oxidic form.

21. The composition of claim 16, wherein the molding according to (a) is obtainable or obtained by a process comprising
(i.1) providing a zeolitic material having an AEI-type framework structure, wherein the zeolitic material has a framework structure comprising Si, a trivalent element X, and oxygen;
(i.2) optionally impregnating the zeolitic material obtained from (i.1) with a source of the one or more alkaline earth metals AEM;
(i.3) preparing a molding comprising the impregnated zeolitic material obtained from (i.2) and optionally a binder material.

22. A process for preparing the composition according to claim 16, the process comprising
(i) providing a molding comprising a zeolitic material having an AEI-type framework structure, wherein the zeolitic material has a framework structure comprising Si, a trivalent element X, and oxygen, wherein the zeolitic material further comprises one or more alkali metals AM and/or one or more alkaline earth metals AEM;
(ii) providing a mixed metal oxide comprising chromium, zinc, and aluminum;
(iii) mixing the molding provided according to (i) with the mixed metal oxide provided according to (ii), obtaining the composition.

23. The process of claim 22, wherein providing a molding according to (i) comprises
(i.1) providing a zeolitic material having an AEI-type framework structure, wherein the zeolitic material has a framework structure comprising Si, a trivalent element X, and oxygen;
(i.2) optionally impregnating the zeolitic material obtained from (i.1) with a source of the one or more alkaline earth metals AEM;
(i.3) preparing a molding comprising the impregnated zeolitic material obtained from (i.2) or the zeolitic material from (i.1) and optionally a binder material.

24. The process of claim 23, wherein preparing a molding according to (i.3) comprises
(i.3.1) preparing a mixture of the impregnated zeolitic material obtained from (i.2) and a source of a binder material;
(i.3.2) subjecting the mixture prepared according to (i.3.1) to shaping.

25. The process of claim 22, wherein providing the mixed metal oxide according to (ii) comprises
(ii.1) co-precipitating a precursor of the mixed metal oxide from sources of the chromium, the zinc, and the aluminum;
(ii.2) washing the precursor obtained from (ii.1);
(ii.3) drying the washed precursor obtained from (ii.2);
(ii.4) calcining the washed precursor obtained from (ii.3).

26. A molding, obtainable or obtained by a process according to claim 23.

27. A mixed metal oxide, obtainable or obtained by a process according to claim 25.

28. A composition, obtainable or obtained by a process according to claim 22.

29. A catalyst or as a catalyst component which comprises the composition according to claim 16.

30. A process for preparing C2 to C4 olefins from a synthesis gas comprising hydrogen and carbon monoxide, the process comprising
(1) providing a gas stream which comprises a synthesis gas stream comprising hydrogen and carbon monoxide;
(2) providing a catalyst comprising a composition according to claim 16;
(3) bringing the gas stream provided in (1) in contact with the catalyst provided in (2), obtaining a reaction mixture stream comprising C2 to C4 olefins.
